(19) United States
(12) Patent Application Publication
(10) Pub. No.: US 2007/0112012 A1
(43) Pub. Date: May 17, 2007

(54) Carbocyclic fused cyclic amines

(76) Inventors: Markus Boehringer, Moehlin (CH); Katrin Groebke Zbinden, Liestal (CH); Wolfgang Haap, Loerrach (DE); Narendra Panday, Muenchen (DE); Fabienne Ricklin, Hombourg (FR); Martin Stahl, Freiburg (DE); Petra Schmitz, Neuenweg (DE)

Correspondence Address: HOFFMANN-LA ROCHE INC.;PATENT LAW DEPARTMENT, 340 KINGSLAND STREET, NUTLEY, NJ, 07110, US

(21) Appl. No.: 11/591,263

(22) Filed: Nov. 1, 2006

(30) Foreign Application Priority Data
Nov. 11, 2005 (EP) 05110635.9

(51) Int. Cl. A61K 31/497; C07D 409/14; C07D 403/02; A61K 31/4709; A61K 31/4439

(52) U.S. Cl. 514/255.05; 546/146; 544/405; 546/277.1; 514/310; 514/337

(57) ABSTRACT

The invention is concerned with novel carbocyclyl fused cyclic amines of formula (I) 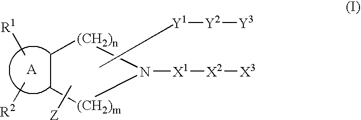
wherein A, X¹ to X³, Y¹ to Y³, Z, R¹, R², m and n are as defined in the description and in the claims, as well as physiologically acceptable salts thereof. These compounds inhibit the coagulation factor Xa and can be used as medicaments.

### PRIORITY TO RELATED APPLICATIONS

This application claims the benefit of European Application No. 05110635.9, filed Nov. 11, 2005, which is hereby incorporated by reference in its entirety.

The invention is concerned with novel carbocyclic fused cyclic amines of formula (I), 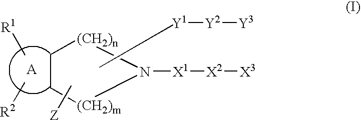
wherein
A is a carbocyclic ring which is a monocyclic or bicyclic aromatic ring of 5 to 12 ring atoms, or a monocyclic or bicyclic non-aromatic ring of 5 to 12 ring atoms, one or two carbon atoms of said carbocyclic ring being optionally replaced with a carbonyl group;
R¹ and R² are independently hydrogen, C₁₋₆alkyl, C₁₋₆ alkoxy, fluoro C₁₋₆ alkoxy, hydroxy C₁₋₆ alkoxy, C₁₋₆ alkoxy C₁₋₆ alkoxy, C₁₋₆ alkoxycarbonyl, mono or di C_{1-6alkyl} substituted amino C₁₋₆ alkoxy, halogen, cyano, nitro, —N(R′)—CO—(C₁₋₆alkyl optionally substituted by one or more fluorine atoms), wherein R′ is hydrogen, C₁₋₆alkyl or fluoro C₁₋₆ alkyl, —N(R′)—CO—O—(C₁₋₆alkyl optionally substituted by one or more fluorine atoms), wherein R′ is hydrogen, C₁₋₆alkyl or fluoro C₁₋₆alkyl, —N(R′)—CO—N(R″) (R′″), wherein R′, R″ and R′″ are independently hydrogen, C₁₋₆alkyl or fluoro C₁₋₆alkyl or —N(R′)—SO₂—(C₁₋₆alkyl optionally substituted by one or more fluorine atoms), wherein R′ is hydrogen, C₁₋₆alkyl or fluoro C₁₋₆alkyl or
R¹ and R² are independently —SO₂—N(R′)(R″), —C(O)—N(R′)(R″) or —N(R′)(R″), wherein R′ and R″ are independently hydrogen, C₁₋₆alkyl or fluoro C₁₋₆alkyl or R′ and R″, together with the nitrogen atom to which they are attached, form heterocycyl;
X¹ is —C(O)—(C₀₋₆alkylene)-NR³—(C₀₋₆alkylene)-, —(C₀₋₆alkylene)-C(O)—NR³—(C₀₋₆alkylene)-, —(C₁₋₆alkylene)-NR³—C(O)—(C₀₋₆alkylene)-, —C(O)—(C₀₋₆alkylene)-, C₀₋₆alkylene, —SO₂—(C₀₋₆alkylene)-, —(C₀₋₆alkylene)-SO₂—NR³—(C₀₋₆alkylene)- or 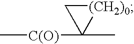
X² is arylene, heteroarylene or heterocyclylene, said arylene, heteroarylene and heterocyclylen being optionally substituted by one or more substituents independently selected from the group consisting of C₁₋₆alkyl, C₁₋₆ alkoxy, halogen, cyano, nitro, amino, —N(R′)—CO—(C₁₋₆alkyl optionally substituted by one or more fluorine atoms), wherein R′ is hydrogen, C₁₋₆alkyl or fluoro C₁₋₆alkyl, —N(R′)—CO—O—(C₁₋₆alkyl optionally substituted by one or more fluorine atoms), wherein R′ is hydrogen, C₁₋₆alkyl or fluoro C₁₋₆ alkyl, —N(R′)—CO—N(R″)(R′″), wherein R′, R″ and R′″ are independently hydrogen, C₁₋₆alkyl or fluoro C₁₋₆alkyl, —C(O)—N(R′)(R″), wherein R′ and R″ are independently hydrogen, C₁₋₆alkyl or fluoro C₁₋₆alkyl, or R′ and R″, together with the nitrogen atom to which they are attached, form heterocycyl, —NR′R″, wherein R′ and R″ are independently hydrogen, C₁₋₆alkyl or fluoro C₁₋₆alkyl, or R′ and R″, together with the nitrogen atom to which they are attached, form heterocycyl, 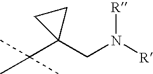
wherein R′ and R″ are independently C₁₋₆alkyl or fluoro C₁₋₆alkyl, or R′ and R″, together with the nitrogen atom to which they are attached, form heterocyclyl, 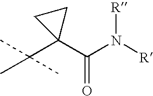
wherein R′ and R″ are independently C₁₋₆alkyl or fluoro C₁₋₆alkyl, or R′ and R″, together with the nitrogen atom to which they are attached, form heterocyclyl, 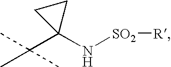
wherein R′ is fluoro C₁₋₆alkyl and 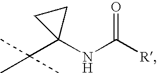
wherein R′ is fluoro C₁₋₆alkyl, and one or two carbon atoms of said arylene, heteroarylene or heterocyclylene being optionally replaced with a carbonyl group;
X³ is hydrogen, aryl, heteroaryl or heterocyclyl, said aryl, heteroaryl and heterocyclyl being optionally substituted by one or more substituents independently selected from the group consisting of C₁₋₆alkyl, C₁₋₆ alkoxy, halogen, cyano, nitro, amino, mono-C₁₋₆alkyl substituted amino, di-C₁₋₆alkyl substituted amino, mono-C₁₋₆alkyl substituted amino-C₁₋₆alkyl, di-C₁₋₆alkyl substituted amino-C₁₋₆alkyl, —SO₂—C₁₋₆alkyl, —SO₂—NH₂, —SO₂—NH—C₁₋₆alkyl and —SO₂—N(C₁₋₆alkyl)₂,
   and one or two carbon atoms of said aryl, heteroaryl and heterocyclyl being optionally replaced with a carbonyl group;
R³ is hydrogen or C₁₋₆alkyl;
Y¹ is —(C₀₋₆alkylene)-C(O)—NR³—(C₀₋₆alkylene)-, —(C₀₋₆alkylene)-NR³—C(O)—(C₀₋₆alkylene)-, —C(O)—(C₀₋₆alkylene)- or C₀₋₆alkylene;
Y² is arylene, heteroarylene or heterocyclylene, said arylene, heteroarylene and heterocyclylene being optionally substituted by one or more substituents independently selected from the group consisting of C₁₋₆alkyl, C₁₋₆ alkoxy, halogen, cyano, nitro, amino, —N(R′)—CO—(C₁₋₆alkyl optionally substituted by one or more fluorine atoms), wherein R′ is hydrogen, C₁₋₆alkyl or fluoro C₁₋₆ alkyl, —N(R′)—CO—O—(C₁₋₆alkyl optionally substituted by one or more fluorine atoms), wherein R′ is hydrogen, C₁₋₆alkyl or fluoro C₁₋₆ alkyl, —N(R′)—CO—N(R″)(R′″), wherein R′, R″ and R′″ are independently hydrogen, C₁₋₆alkyl or fluoro C₁₋₆alkyl, —C(O)—N(R′)(R″), wherein R′ and R″ are independently hydrogen, C₁₋₆alkyl or halo C₁₋₆alkyl, or R′ and R″, together with the nitrogen atom to which they are attached, form heterocycyl, —NR′R″, wherein R′ and R″ are independently hydrogen, C₁₋₆alkyl or halo C₁₋₆alkyl, or R′ and R″, together with the nitrogen atom to which they are attached, form heterocycyl, 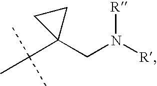
wherein R′ and R″ are independently C₁₋₆alkyl or fluoro C₁₋₆alkyl, or R′ and R″, together with the nitrogen atom to which they are attached, form heterocyclyl, 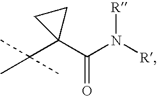
wherein R′ and R″ are independently C₁₋₆alkyl or fluoro C₁₋₆alkyl, or R′ and R″, together with the nitrogen atom to which they are attached, form heterocyclyl, 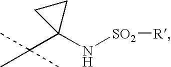
wherein R′ is fluoro C₁₋₆alkyl and 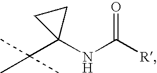
wherein R′ is C₁₋₆alkyl,
   and one or two carbon atoms of said arylene, heteroarylene or heterocyclylene being optionally replaced with a carbonyl group;
Y³ is hydrogen, aryl, heteroaryl or heterocyclyl, said aryl, heteroaryl and heterocyclyl being optionally substituted by one or more substituents independently selected from the group consisting of C₁₋₆alkyl, C₁₋₆ alkoxy, halogen, cyano, nitro, amino, mono-C₁₋₆alkyl substituted amino, di-C₁₋₆alkyl substituted amino, mono-C₁₋₆alkyl substituted amino-C₁₋₆alkyl, di-C₁₋₆alkyl substituted amino-C₁₋₆alkyl, —SO₂—C₁₋₆alkyl, —SO₂—NH₂, —SO₂—NH—C₁₋₆alkyl and —SO₂—N(C₁₋₆alkyl)₂, and one or two carbon atoms of said aryl, heteroaryl and heterocyclyl being optionally replaced with a carbonyl group;
Z is attached to the same carbon atom as —Y¹—Y²—Y³, and hydrogen or C₁₋₆alkyl;
n is 0, 1 or 2;
m is 0, 1 or 2;
m+n is 2 or 3;
o is an integer from 1 to 5;
   and prodrugs and pharmaceutically acceptable salts thereof.

Further, the invention is concerned with a process and an intermediate for the manufacture of the above compounds, pharmaceutical preparations which contain such compounds, the use of these compounds for the production of pharmaceutical preparations as well as a process for the manufacture of the intermediate.

The compounds of formula (I) are active compounds and inhibit the coagulation factor Xa. These compounds consequently influence blood coagulation. They therefore inhibit the formation of thrombin and can be used for the treatment and/or prevention of thrombotic disorders, such as amongst others, arterial and venous thrombosis, deep vein thrombosis, peripheral arterial occlusive disease (PAOD), unstable angina pectoris, myocardial infarction, coronary artery disease, pulmonary embolism, stroke (cerebral thrombosis) due to atrial fibrillation, inflammation and arteriosclerosis. They have potentially benefit in the treatment of acute vessel closure associated with thrombolytic therapy and restenosis, e.g. after transluminal coronary angioplasty (PTCA) or bypass grafting of the coronary or peripheral arteries and in the maintenance of vascular access patency in long term hemodialysis patients. F.Xa inhibitors of this invention may form part of a combination therapy with an anticoagulant with a different mode of action or with a platelet aggregation inhibitor or with a thrombolytic agent. Furthermore, these compounds have an effect on tumour cells and prevent metastases. They can therefore also be used as antitumour agents.

Other inhibitors of factor Xa had previously been suggested for the inhibition of the formation of thrombin and for the treatment of related diseases. However, there is still a need for novel factor Xa inhibitors which exhibit improved pharmacological properties, e.g. an improved selectivity towards thrombin.

The present invention provides novel compounds of formula (I) which are factor Xa inhibitors. The compounds of the present invention unexpectedly inhibit coagulation factor Xa and also exhibit improved pharmacological properties compared to other compounds already known in the art.

Unless otherwise indicated, the following definitions are set forth to illustrate and define the meaning and scope of the various terms used to describe the invention herein.

The term “halogen” or “halo” means fluorine, chlorine, bromine and iodine, with fluorine, chlorine and bromine being preferred, and fluorine and chlorine being more preferred.

The term “C₁₋₆alkyl”, alone or in combination with other groups, means a branched or straight-chain monovalent alkyl radical, having one to six carbon atoms. This term is further exemplified by such radicals as methyl, ethyl, n-propyl, isopropyl, n-butyl, s-butyl, t-butyl. C₁₋₄alkyl is more preferred.

The term “C₀₋₆alkylene” means a linear saturated divalent hydrocarbon radical of one to six carbon atoms or a branched divalent hydrocarbon radical of three to six carbon atoms or a bond when C is 0, e.g., methylene, ethylene, 2,2-dimethylethylene, propylene.

The term “C₁₋₆ alkoxy”, alone or in combination with other groups, means the group R′—O—, wherein R′ is a C₁₋₆alkyl.

The term “hydroxy C₁₋₆ alkoxy” means C₁₋₆ alkoxy substituted by one or more hydroxy group.

The term “fluoro C₁₋₆alkyl” or “fluoro C₁₋₆ alkoxy” means C₁₋₆alkyl or C₁₋₆ alkoxy substituted by one or more fluorine atoms, preferably one to three fluorine atoms.

The term “aryl” means phenyl or naphthyl. Phenyl is preferred.

The term “arylene”, alone or in combination with other groups, means a divalent aryl group as defined above. 1,4-phenylene is preferred.

The term “heterocyclyl”, alone or combination with other groups, means non-aromatic mono- or bi-cyclic radicals of three to eight ring atoms wherein one or two ring atoms are heteroatoms selected from N, O, or S(O)ₙ (where n is an integer from 0 to 2), the remaining ring atoms being C. Monocyclic radicals are preferred.

The term “heterocyclylene”, alone or combination with other groups, means a divalent heterocyclyl group as defined above.

The term “heteroaryl”, alone or combination with other groups, means a monocyclic or bicyclic aromatic radical of 5 to 12 ring atoms, containing one, two, or three ring heteroatoms selected from N, O, and S, the remaining ring atoms being C, one or two carbon atoms of said ring being optionally replaced with a carbonyl group, with the understanding that the attachment point of the heteroaryl radical will be on an aromatic ring. Monocyclic radicals are preferred.

The term “heteroarylene”, alone or combination with other groups, means a divalent heteroaryl group as defined above.

The term “bicyclic aromatic ring” or “bicyclic aromatic radical” contains both an aromatic monocyclic ring fused by another aromatic monocyclic ring and an aromatic monocyclic ring fused by a non-aromatic monocyclic ring. When the term “bicyclic aromatic ring” or “bicyclic aromatic radical” is used in the context of the definition of “heteroaryl” or “heteroaryl ring”, at least one heteroatom must exist in the aromatic ring as a ring member. When the heteroaryl ring as A ring in formula I is a bicyclic aromatic ring, and this bicyclic aromatic ring is an aromatic monocyclic ring fused by a non-aromatic monocyclic ring, then the aromatic ring is directly fused to the nitrogen containing ring to which —Y¹—Y²—Y³, —X¹—X²—X³ and Z are attached.

Preferred radicals for the chemical groups whose definitions are given above are those specifically exemplified in Examples.

Compounds of formula (I) can form pharmaceutically acceptable acid addition salts. Examples of such pharmaceutically acceptable salts are salts of compounds of formula (I) with physiologically compatible mineral acids, such as hydrochloric acid, sulphuric acid, sulphurous acid or phosphoric acid; or with organic acids, such as methanesulphonic acid, p-toluenesulphonic acid, acetic acid, lactic acid, trifluoroacetic acid, citric acid, fumaric acid, maleic acid, tartaric acid, succinic acid or salicylic acid. The term “pharmaceutically acceptable salts” refers to such salts. Compounds of formula (I) wherein a COOH group is present can further form salts with bases. Examples of such salts are alkaline, earth-alkaline and ammonium salts such as e.g. Na—, K—, Ca— and trimethylammoniumsalt. The term “pharmaceutically acceptable salts” also refers to such salts. Acid addition salts as described above are preferred.

“Optional” or “optionally” means that the subsequently described event or circumstance may but need not occur, and that the description includes instances where the event or circumstance occurs and instances wherein it does not. For example, “aryl group optionally substituted with an alkyl group” means that the alkyl may but need not be present, and the description includes situations where the aryl group is substituted with an alkyl group and situations where the aryl group is not substituted with the alkyl group.

“Pharmaceutically acceptable excipient” means an excipient that is useful in preparing a pharmaceutical composition that is generally safe, non-toxic and neither biologically nor otherwise undesirable, and includes excipient that is acceptable for veterinary use as well as human pharmaceutical use. A “pharmaceutically acceptable excipient” as used in the specification and claims includes both one and more than one such excipient.

Compounds that have the same molecular Formula but differ in the nature or sequence of bonding of their atoms or the arrangement of their atoms in space are termed “isomers.” Isomers that differ in the arrangement of their atoms in space are termed “stereoisomers”. Stereoisomers that are not mirror images of one another are termed “diastereomers” and those that are non-superimposable mirror images of each other are termed “enantiomers”. When a compound has an asymmetric center, for example, if a carbon atom is bonded to four different groups, a pair of enantiomers is possible. An enantiomer can be characterized by the absolute configuration of its asymmetric center and is described by the R- and S-sequencing rules of Cahn, Ingold and Prelog, or by the manner wherein the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture”.

The compounds of formula (I) can possess one or more asymmetric centers. Unless indicated otherwise, the description or naming of a particular compound in the specification and claims is intended to include both individual enantiomers and mixtures, racemic or otherwise, thereof. The methods for the determination of stereochemistry and the separation of stereoisomers are well-known in the art (see discussion in Chapter 4 of “Advanced Organic Chemistry”, 4th edition J. March, John Wiley and Sons, New York, 1992).

While the broadest definition of this invention is described before, certain compounds of formula (I) are preferred.

i) A preferred compound of the invention is a compound of formula (I), wherein A is a benzene ring or cyclohexane ring.

ii) Another preferred compound of the invention is a compound of formula (I), wherein X¹ is —C(O)—(C₀₋₆alkylene)-NR³—(C₀₋₆alkylene)-, wherein R³ is as defined before. X¹ is preferably —(C₀₋₆alkylene)-C(O)—NH—, and more preferably —C(O)—NH—.

iii) Another preferred compound of the invention is a compound of formula (I), wherein X² is arylene or heteroarylene, said arylene and heteroarylene being optionally substituted by one or more substituents independently selected from the group consisting of C₁₋₆ alkoxy and halogen, and X³ is hydrogen. Preferably —X²—X³ forms phenyl or pyridyl, said phenyl and pyridyl being optionally substituted by one or more same or different halogen atoms. More preferably —X²—X³ forms 4-chlorophenyl or 5-chloropyridin-2-yl.

iv) Another preferred compound of the invention is a compound of formula (I) wherein X² is 1,4-phenylene optionally substituted by one or more same or different halogen atoms, preferably 1,4-phenylene optionally substituted by one or more fluorine atoms, more preferably 2-fluoro-1,4 phenylene.

v) Another preferred compound of the invention is a compound of formula (I) wherein X³ is heteroaryl optionally substituted by one or more substituents independently selected from the group consisting of C₁₋₆alkyl, C₁₋₆ alkoxy, halogen, cyano, nitro, amino, mono-C₁₋₆alkyl substituted amino, di-C₁₋₆alkyl substituted amino, mono-C₁₋₆alkyl substituted amino-C₁₋₆alkyl, di-C₁₋₆alkyl substituted amino-C₁₋₆alkyl, —SO₂—C₁₋₆alkyl, —SO₂—NH₂, —SO₂—NH—C₁₋₆alkyl and —SO₂—N(C₁₋₆alkyl)₂, and one or two carbon atoms of said heteroaryl being optionally replaced with a carbonyl group. Preferably X³ is unsubstituted heteroaryl which is a monocyclic aromatic ring of 5 or 6 ring atoms, containing one or two, preferably one ring nitrogen atom, and one carbon atom of said heteroaryl being optionally replaced with a carbonyl group. Preferably the ring nitrogen atom of the heteroaryl is directly attached to X², and one of the ring carbon atoms next to said ring nitrogen atom is replaced with a carbonyl group. X³ is especially 2-oxo-2H-pyridin-1-yl.

vi) Another preferred compound of the invention is a compound of formula (I), wherein Y¹ is —C(O)—(C₀₋₆alkylene)-NR³—(C₀₋₆alkylene)-, —(C₀₋₆alkylene)-NR³—C(O)—(C₀₋₆alkylene)- or —C(O)—(C₀₋₆alkylene)-, wherein R³ is as defined before. Y¹ is preferably —C(O)—NH—, —C(O)— or —CH₂—NH—C(O)—, and more preferably —C(O)—NH—.

vii) Another preferred compound of the invention is a compound of formula (I), wherein Y² is arylene or heteroarylene, said arylene and heteroarylene being optionally substituted by one or more same or different halogen atoms and Y³ is hydrogen. Preferably —Y²—Y³ forms phenyl or thienyl, said phenyl and thienyl being optionally substituted by one or more same or different halogen atoms. More preferably —Y²—Y³ forms 5-chloro-2-thienyl.

viii) Another preferred compound of the invention is a compound of formula (I) wherein Y² is 1,4-phenylene optionally substituted by one or more same or different halogen atoms, preferably 1,4-phenylene optionally substituted by one or more fluorine atoms, more preferably 2-fluoro-1,4 phenylene.

ix) Another preferred compound of the invention is a compound of formula (I) wherein Y³ is heteroaryl optionally substituted by one or more substituents independently selected from the group consisting of C₁₋₆alkyl, C₁₋₆ alkoxy, halogen, cyano, nitro, amino, mono-C₁₋₆alkyl substituted amino, di-C₁₋₆alkyl substituted amino, mono-C₁₋₆alkyl substituted amino-C₁₋₆alkyl, di-C₁₋₆alkyl substituted amino-C₁₋₆alkyl, —SO₂—C₁₋₆alkyl, —SO₂—NH₂, —SO₂—NH—C₁₋₆alkyl and —SO₂—N(C₁₋₆alkyl)₂, and one or two carbon atoms of said heteroaryl being optionally replaced with a carbonyl group. Preferably Y³ is unsubstituted heteroaryl which is a monocyclic aromatic ring of 5 or 6 ring atoms, containing one or two, preferably one ring nitrogen atom, and one carbon atom of said heteroaryl being optionally replaced with a carbonyl group. Preferably the ring nitrogen atom of the heteroaryl is directly attached to Y², and one of the ring carbon atoms next to said ring nitrogen atom is replaced with a carbonyl group. Y³ is especially 2-oxo-2H-pyridyn-1-yl.

x) Another preferred compound of the invention is a compound of formula (I) wherein only one of X³ and Y³ is hydrogen.

xi) Another preferred compound of the invention is a compound of formula (I) wherein one of R¹ and R² is hydrogen, and the other is hydrogen, C₁₋₆alkyl, C₁₋₆ alkoxycarbonyl, C₁₋₆ alkoxy or —C(O)—N(R′)(R″), wherein R′ and R″ are independently hydrogen or C₁₋₆alkyl.

xii) Another preferred compound of the invention is a compound of formula (I) wherein Z is hydrogen or methyl.

xiii) Another preferred compound of the invention is a compound of formula (I) which is 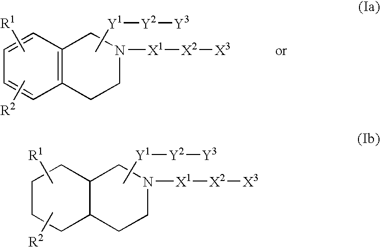
wherein X¹, X², X³, Y¹, Y², Y³, R¹ and R² are as defined before.

a) A preferred compound in group xiii) is a compound wherein X¹ is —(C₀₋₆alkylene)-C(O)—NH—.

b) Another preferred compound in group xiii) is a compound wherein X¹ is —C(O)—NH—.

c) Another preferred compound in group xiii) is a compound, wherein X² is arylene or heteroarylene, said arylene and heteroarylene being optionally substituted by one or more substituents independently selected from the group consisting of C₁₋₆ alkoxy and halogen, and X³ is hydrogen.

d) Another preferred compound in group xiii) is a compound, wherein —X²—X³ forms phenyl or pyridyl, said phenyl and pyridyl being optionally substituted by one or more same or different halogen atoms.

e) Another preferred compound in group xiii) is a compound, wherein —X²—X³ forms 4-chlorophenyl.

f) Another preferred compound in group xiii) is a compound, wherein Y¹ is —(C₀₋₆alkylene)-C(O)—NH—.

g) Another preferred compound in group xiii) is a compound, wherein Y¹ is —C(O)—NH—.

h) Another preferred compound in group xiii) is a compound, wherein Y² is 1,4-phenylene optionally substituted by one or more same or different halogen atoms.

i) Another preferred compound in group xiii) is a compound, wherein Y² is 2-fluoro-1,4 phenylene.

j) Another preferred compound in group xiii) is a compound, wherein Y³ is heteroaryl or heterocyclyl, said heteroaryl and heterocyclyl being optionally substituted by one or more substituents independently selected from the group consisting of C₁₋₆alkyl, C₁₋₆ alkoxy, halogen, cyano, nitro, amino, mono-C₁₋₆alkyl substituted amino, di-C₁₋₆alkyl substituted amino, mono-C₁₋₆alkyl substituted amino-C₁₋₆alkyl, di-C₁₋₆alkyl substituted amino-C₁₋₆alkyl, —SO₂—C₁₋₆alkyl, —SO₂—NH₂, —SO₂—NH—C₁₋₆alkyl and —SO₂—N(C₁₋₆alkyl)₂, and one or two carbon atoms of said heteroaryl and heterocyclyl being optionally replaced with a carbonyl group.

k) Another preferred compound in group xiii) is a compound, wherein Y³ is 2-oxo-2H-pyridyn-1-yl.

l) Another preferred compound in group xiii) is a compound, wherein —Y¹—Y²—Y³ is bonded to 3 position of the isoquinoline ring.

m) Another preferred compound in group xiii) is a compound, wherein R¹ and R² are hydrogen.

n) Another preferred compound in group xiii) is a compound, which is 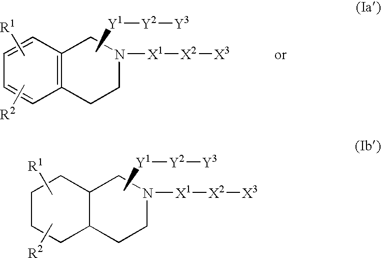
wherein X¹, X², X³, Y¹, Y², Y³, R¹ and R² are as defined before.

xiv) Another preferred compound of the invention is a compound of formula (I) which is 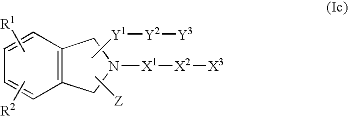
wherein X¹, X², X³, Y¹, Y², Y³, R¹, R² and Z are as defined before.

a) A preferred compound in group ix) is a compound, wherein, wherein X¹ is —(C₀₋₆alkylene)-C(O)—NH—.

b) Another preferred compound in group ix) is a compound, wherein X¹ is —C(O)—NH—.

c) Another preferred compound in group ix) is a compound, wherein X² is arylene or heteroarylene, said arylene and heteroarylene being optionally substituted by one or more substituents independently selected from the group consisting of C₁₋₆ alkoxy and halogen, and X³ is hydrogen.

d) Another preferred compound in group ix) is a compound, wherein —X²—X³ forms phenyl or pyridyl, said phenyl and pyridyl being optionally substituted by one or more same or different halogen atoms.

e) Another preferred compound in group ix) is a compound, wherein —X²—X³ forms 4-chlorophenyl or 5-chloro-2-pyridyl.

f) Another preferred compound in group ix) is a compound, wherein Y¹ is —(C₀₋₆alkylene)-C(O)—NH—.

g) Another preferred compound in group ix) is a compound, wherein Y¹ is —C(O)—NH—.

h) Another preferred compound in group ix) is a compound, wherein Y² is 1,4-phenylene optionally substituted by one or more same or different halogen atoms.

i) Another preferred compound in group ix) is a compound, wherein Y² is 2-fluoro-1,4 phenylene.

j) Another preferred compound in group ix) is a compound, wherein Y³ is heteroaryl or heterocyclyl, said heteroaryl and heterocyclyl being optionally substituted by one or more substituents independently selected from the group consisting of C₁₋₆alkyl, C₁₋₆ alkoxy, halogen, cyano, nitro, amino, mono-C₁₋₆alkyl substituted amino, di-C₁₋₆alkyl substituted amino, mono-C₁₋₆alkyl substituted amino-C₁₋₆alkyl, di-C₁₋₆alkyl substituted amino-C₁₋₆alkyl, —SO₂—C₁₋₆alkyl, —SO₂—NH₂, —SO₂—NH—C₁₋₆alkyl and —SO₂—N(C₁₋₆alkyl)₂, and one or two carbon atoms of said heteroaryl and heterocyclyl being optionally replaced with a carbonyl group.

k) Another preferred compound in group ix) is a compound, wherein Y³ is 2-oxo-2H-pyridyn-1-yl.

l) Another preferred compound in group ix) is a compound, wherein —Y¹—Y²—Y³ is bonded to 1 position of the isoindole ring.

m) Another preferred compound in group ix) is a compound, wherein R¹ and R² are hydrogen.

n) Another preferred compound in group ix) is a compound, wherein Z is hydrogen or methyl.

o) Another preferred compound in group ix) is a compound, which is 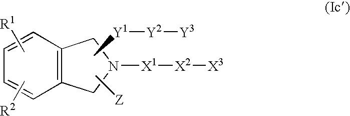
wherein X¹, X², X³, Y¹, Y², Y³, R¹, R² and Z are as defined before.

xv) Another preferred compound of the invention is a compound of formula (I) which is 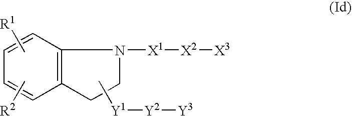
wherein X¹, X², X³, Y¹, Y², Y³, R¹ and R² are as defined before.

a) A preferred compound in group x) is a compound, wherein X¹ is —(C₀₋₆alkylene)-C(O)—NH—.

b) Another preferred compound in group x) is a compound, wherein X¹ is —C(O)—NH—.

c) Another preferred compound in group x) is a compound, wherein X² is 1,4-phenylene optionally substituted by one or more same or different halogen atoms.

d) Another preferred compound in group x) is a compound, wherein X² is 2-fluoro-1,4 phenylene.

e) Another preferred compound in group x) is a compound, wherein X³ is heteroaryl which is optionally substituted by one or more substituents independently selected from the group consisting of C₁₋₆alkyl, C₁₋₆ alkoxy, halogen, cyano, nitro, amino, mono-C₁₋₆alkyl substituted amino, di-C₁₋₆alkyl substituted amino, mono-C₁₋₆alkyl substituted amino-C₁₋₆alkyl, di-C₁₋₆alkyl substituted amino-C₁₋₆alkyl, —SO₂—C₁₋₆alkyl, —SO₂—NH₂, —SO₂—NH—C₁₋₆alkyl and —SO₂—N(C₁₋₆alkyl)₂, and one or two carbon atoms of said heteroaryl being optionally replaced with a carbonyl group.

f) Another preferred compound in group x) is a compound, wherein X³ is 2-oxo-2H-pyridyn-1-yl.

g) Another preferred compound in group x) is a compound, wherein Y¹ is —(C₀₋₆alkylene)-NH—C(O)—.

h) Another preferred compound in group x) is a compound, wherein Y¹ is —CH₂—NH—C(O)—.

i) Another preferred compound in group x) is a compound, wherein Y² is heteroarylene which is optionally substituted by one or more same or different halogen atoms, and Y³ is hydrogen.

j) Another preferred compound in group x) is a compound, wherein —Y²—Y³ forms thienyl optionally substituted by one or more same or different halogen atoms.

k) Another preferred compound in group x) is a compound, wherein —Y²—Y³ forms 5-chloro-2-thienyl.

l) Another preferred compound in group x) is a compound, wherein Y¹—Y²—Y³ is bonded to 3 position of the indole ring.

m) Another preferred compound in group x) is a compound, wherein one of R¹ and R² is hydrogen or C₁₋₆ alkoxy, and the other is selected from the group consisting of hydrogen, C₁₋₆alkyl, C₁₋₆ alkoxy, C₁₋₆ alkoxycarbonyl, halogen and —C(O)—N(R′)(R″), wherein R′ and R″ are independently hydrogen, C₁₋₆alkyl or fluoro C₁₋₆alkyl.

n) Another preferred compound in group x) is a compound, which is 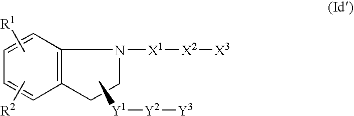
wherein X¹, X², X³, Y¹, Y², Y³, R¹ and R² are as defined before.

xvi) Another preferred compound of the invention is a compound of formula (I) which is 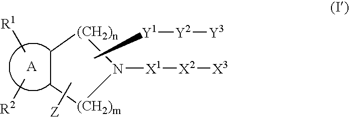
wherein A, X¹ to X³, Y¹ to Y³, Z, R¹, R², m and n are as defined before.

xvii) Particularly preferred compounds of the present invention are:
1,3-Dihydro-isoindole-1,2-dicarboxylic acid 2-[(4-chloro-phenyl)-amide]1-{[2-fluoro-4-(2-oxo-2H-pyridin-1-yl)-phenyl]-amide},
1,3-Dihydro-isoindole-1,2-dicarboxylic acid 2-[(5-chloro-pyridin-2-yl)-amide]1-{[2-fluoro-4-(2-oxo-2H-pyridin-1-yl)-phenyl]-amide},
(S)-1,3-Dihydro-isoindole-1,2-dicarboxylic acid 2-[(4-chloro-phenyl)-amide]1-{[4-(2-oxo-2H-pyridin-1-yl)-phenyl]-amide},
(R)-1,3-Dihydro-isoindole-1,2-dicarboxylic acid 2-[(4-chloro-phenyl)-amide]1-{[4-(2-oxo-2H-pyridin-1-yl)-phenyl]-amide},
(R)-1,3-Dihydro-isoindole-1,2-dicarboxylic acid 2-[(5-chloro-pyridin-2-yl)-amide]1-{[4-(2-oxo-2H-pyridin-1-yl)-phenyl]-amide},
(S)-1,3-Dihydro-isoindole-1,2-dicarboxylic acid 2-[(5-chloro-pyridin-2-yl)-amide]1-{[4-(2-oxo-2H-pyridin-1-yl)-phenyl]-amide},
(S)-1,3-Dihydro-isoindole-1,2-dicarboxylic acid 2-[(4-chloro-phenyl)-amide]1-{[4-(2-oxo-2H-pyrazin-1-yl)-phenyl]-amide},
(R)-1,3-Dihydro-isoindole-1,2-dicarboxylic acid 2-[(4-chloro-phenyl)-amide]1-{[4-(2-oxo-2H-pyrazin-1-yl)-phenyl]-amide},
1,3-Dihydro-isoindole-1,2-dicarboxylic acid 2-[(4-chloro-phenyl)-amide]1-{[2-fluoro-4-(2-oxo-2H-pyrazin-1-yl)-phenyl]-amide},
1,3-Dihydro-isoindole-1,2-dicarboxylic acid 2-[(4-chloro-phenyl)-amide]1-{[2-fluoro-4-(3-oxo-morpholin-4-yl)-phenyl]-amide},
1-Methyl-1,3-dihydro-isoindole-1,2-dicarboxylic acid 2-[(4-chloro-phenyl)-amide]1-{[2-fluoro-4-(2-oxo-2H-pyridin-1-yl)-phenyl]-amide},
(R)-1-Methyl-1,3-dihydro-isoindole-1,2-dicarboxylic acid 2-[(4-chloro-phenyl)-amide]1-{[2-fluoro-4-(2-oxo-2H-pyridin-1-yl)-phenyl]-amide},
1,3-Dihydro-isoindole-1,2,6-tricarboxylic acid 2-[(4-chloro-phenyl)-amide]6-dimethylamide 1-{[2-fluoro-4-(2-oxo-2H-pyridin-1-yl)-phenyl]-amide},
(R)-3,4-Dihydro-1H-isoquinoline-2,3-dicarboxylic acid 2-[(4-chloro-phenyl)-amide]3-{[2-fluoro-4-(2-oxo-2H-pyridin-1-yl)-phenyl]-amide},
(S)-3,4-Dihydro-1H-isoquinoline-2,3-dicarboxylic acid 2-[(4-chloro-phenyl)-amide]3-{[2-fluoro-4-(2-oxo-2H-pyridin-1-yl)-phenyl]-amide},
(R)-3,4-Dihydro-1H-isoquinoline-2,3-dicarboxylic acid 2-[(4-chloro-phenyl)-amide]3-{[2-fluoro-4-(3-oxo-morpholin-4-yl)-phenyl]-amide},
(R)-3,4-Dihydro-1H-isoquinoline-2,3-dicarboxylic acid 2-[(5-chloro-pyridin-2-yl)-amide]3-{[2-fluoro-4-(2-oxo-2H-pyridin-1-yl)-phenyl]-amide},
(R)-3,4-Dihydro-1H-isoquinoline-2,3-dicarboxylic acid 3-{[2-fluoro-4-(2-oxo-2H-pyridin-1-yl)-phenyl]-amide}2-[(4-methoxy-phenyl)-amide],
(R)-3,4-Dihydro-1H-isoquinoline-2,3-dicarboxylic acid 3-[(4-chloro-phenyl)-amide]2-{[2-fluoro-4-(2-oxo-2H-pyridin-1-yl)-phenyl]-amide},
(R)-Octahydro-isoquinoline-2,3-dicarboxylic acid 2-[(4-chloro-phenyl)-amide]3-{[2-fluoro-4-(2-oxo-2H-pyridin-1-yl)-phenyl]-amide},
(R)-3-{[(5-Chloro-thiophene-2-carbonyl)-amino]-methyl}-2,3-dihydro-indole-1-carboxylic acid [2-fluoro-4-(2-oxo-2H-pyridin-1-yl)-phenyl]-amide,
(S)-3-{[(5-Chloro-thiophene-2-carbonyl)-amino]-methyl}-2,3-dihydro-indole-1-carboxylic acid [2-fluoro-4-(2-oxo-2H-pyridin-1-yl)-phenyl]-amide,
3-{[(5-Chloro-thiophene-2-carbonyl)-amino]-methyl}-1-[2-fluoro-4-(2-oxo-2H-pyridin-1-yl)-phenylcarbamoyl]-2,3-dihydro-1H-indole-6-carboxylic acid methyl ester,
3-{[(5-Chloro-thiophene-2-carbonyl)-amino]-methyl}-2,3-dihydro-indole-1,6-dicarboxylic acid 6-dimethylamide 1-{[2-fluoro-4-(2-oxo-2H-pyridin-1-yl)-phenyl]-amide},
5-Chloro-thiophene-2-carboxylic acid (1-{[2-fluoro-4-(2-oxo-2H-pyridin-1-yl)-phenylcarbamoyl]-methyl}-2,3-dihydro-1H-indol-3-yl)-amide,
3-{[(5-Chloro-thiophene-2-carbonyl)-amino]-methyl}-4-methyl-2,3-dihydro-indole-1-carboxylic acid [2-fluoro-4-(2-oxo-2H-pyridin-1-yl)-phenyl]-amide, and
4-Chloro-3-{[(5-chloro-thiophene-2-carbonyl)-amino]-methyl}-2,3-dihydro-indole-1-carboxylic acid [2-fluoro-4-(2-oxo-2H-pyridin-1-yl)-phenyl]-amide.

The compounds of the present invention can be prepared, for example, by the general synthetic procedures described below.

General Synthetic Procedures

Abbreviations

AcOEt: Ethyl acetate

AIBN: 2,2′-Azobis-(2-methyl-propionitrile)

Boc₂O: Di-tert-butyl-dicarbonate

BOP: Benzotriazolyl-N-oxy-tris(dimethylamino)-phosphonium hexafluorophosphate

BOP-Cl: Bis-(2-oxo-3-oxazolidinyl)-phosphinic acid chloride

tBuOMe: t-Butyldimethylether

DIPEA: Diisopropyl ethyl amine

DMA: N,N-Dimethylacetamide

DMAP: 4-Dimethylaminopyridine

DME: 1,2-Dimethoxyethane

DMF: N,N-Dimethylformamide

DMSO: Dimethylsulfoxide

EDCI: N-(3-Dimethylaminopropyl)-N′-ethyl-carbodiimide hydrochloride

HATU: 1-[Bis(dimethylamino)methylene]-1H-1,2,3-triazolo [4,5-b]pyridinium 3-oxide, hexafluorophosphate

HOBT: 1-Hydroxybenzotriazole

MeOH: Methanol

TEA: Triethylamine

TFA: Trifluoroacetic acid

THF: Tetrahydrofuran

General Procedure

Amidation: The intermediate carboxylic acid is reacted with an amine H₂NY²Y³ in a suitable solvent such as CH₂Cl₂, DMF, acetonitrile, THF. Activation is effected by an amide coupling reagent such as BOP, BOP-Cl, HATU/HOBT, EDCI/DMAP in the presence of a base like TEA, DIPEA, N-methylmorpholine etc. at 0° C. to 50° C. Reaction times ranged from 1 hr-72 hrs. Preferred conditions are DMF, BOPCl and DIPEA.

Deprotection: The intermediate is treated with a mineral acid such as HCl, HBr, H₂SO₄ or H₃PO₄ or a carbonic acid, in a solvent such as CH₂Cl₂, dioxane or HOAc at 0 to 60° C. Preferred conditions are 4N HCl in dioxane.

Acylation: The intermediate is reacted with a substituted phenyl-isocyanate or substituted p-nitrophenylcarbamate in a suitable solvent such as dichloromethane, DMF, DMSO, THF at 0 to 120° C.
Indoline Derivatives 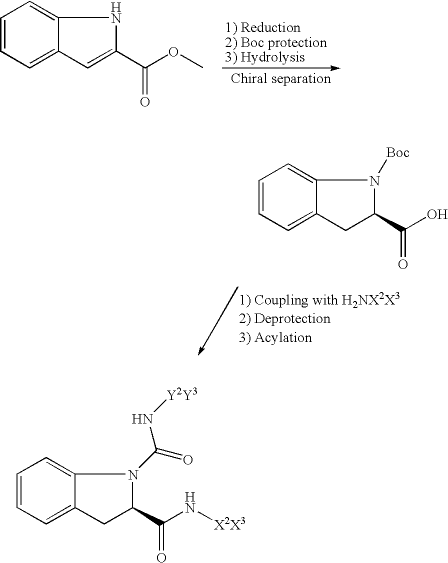
X², X³, Y² and Y³ are as defined before.
Isoindoline Derivatives

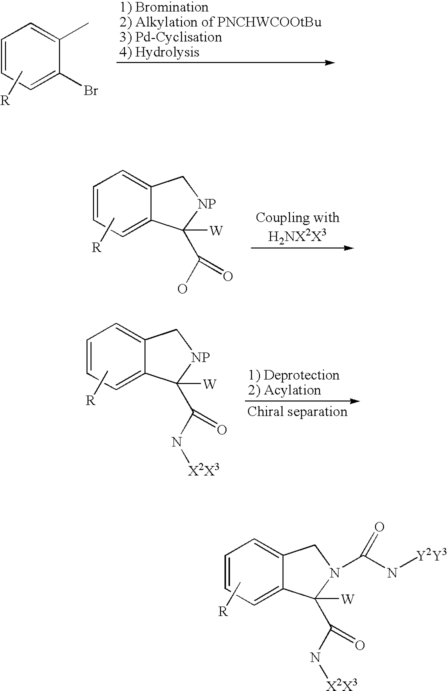

X², X³, Y² and Y³ are as defined before. P is an amino protecting group such as t-butoxycarbonyl or benzyl. R is hydrogen or amide. W is hydrogen or methyl.
Tetrahydroisoquinoline Derivatives

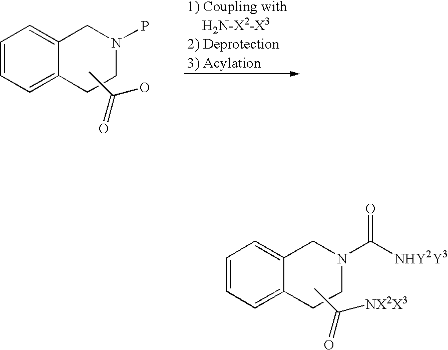

X², X³, Y² and Y³ are as defined before. P is an amino protecting group such as t-butoxycarbonyl or benzyl.
Saturated Bicyclic Derivatives

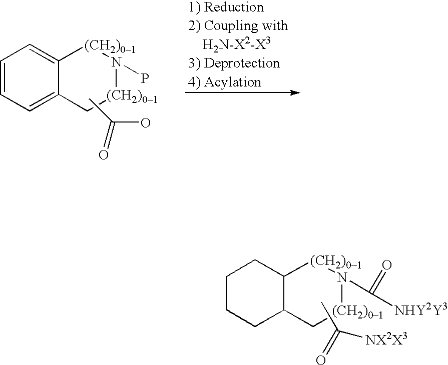

X², X³, Y² and Y³ are as defined before. P is an amino protecting group such as t-butoxycarbonyl or benzyl.
1,3-Amino(methyl)-indoline Derivatives

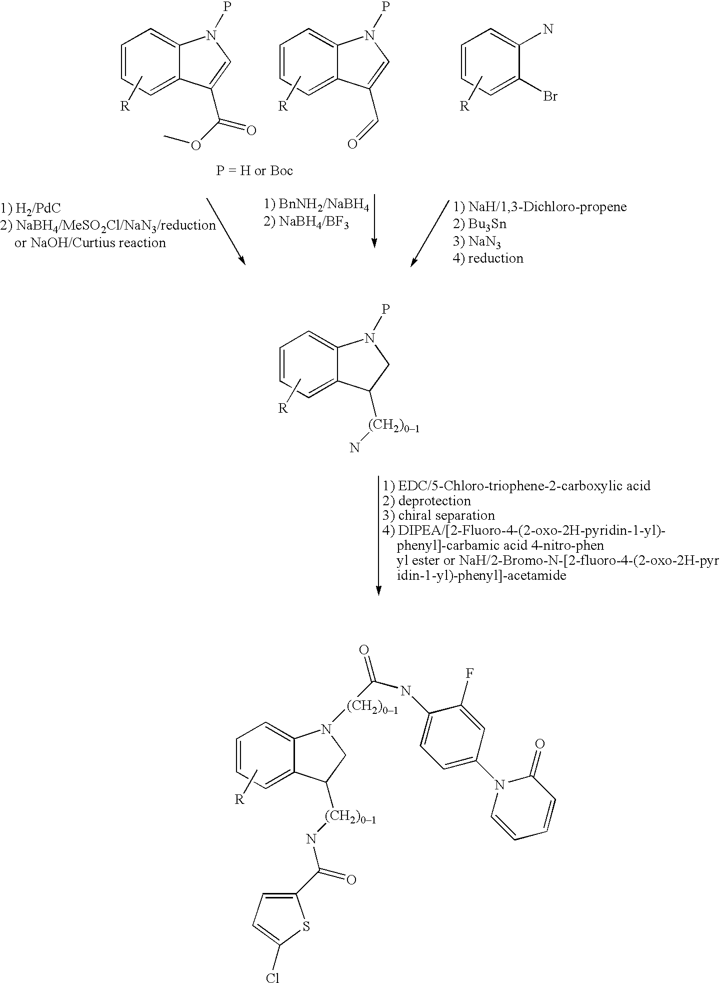

R is hydrogen, methyl, methoxy, halogene or amide. P is an amino protecting group such as t-butoxycarbonyl or benzyl.

As described above, the compounds of formula (I) are active compounds and inhibit the coagulation factor Xa. These compounds consequently influence both platelet activation which is induced by this factor and plasmatic blood coagulation. They therefore inhibit the formation of thrombi and can be used for the treatment and/or prevention of thrombotic disorders, such as, amongst others, arterial and venous thrombosis, deep vein thrombosis, peripheral arterial occlusive disease (PAOD), unstable angina pectoris, myocardial infarction, coronary artery disease, pulmonary embolism, stroke (cerebral thrombosis) due to atrial fibrillation, inflammation and arteriosclerosis. The compounds of the present invention can also be used in the treatment of acute vessel closure associated with thrombolytic therapy and restenosis, e.g. after transluminal coronary angioplasty (PTCA) or bypass grafting of the coronary or peripheral arteries and in the maintenance of vascular access patency in long term hemodialysis patients. F.Xa inhibitors of this invention may form part of a combination therapy with an anticoagulant with a different mode of action or with a platelet aggregation inhibitor or with a thrombolytic agent. Furthermore, these compounds have an effect on tumour cells and prevent metastases. They can therefore also be used as antitumour agents.

Prevention and/or treatment of thrombotic disorders, particularly arterial or deep vein thrombosis, is the preferred indication.

The invention therefore also relates to pharmaceutical compositions comprising a compound as defined above and a pharmaceutically acceptable excipient.

The invention likewise embraces compounds as described above for use as therapeutically active substances, especially as therapeutically active substances for the treatment and/or prophylaxis of diseases which are associated with the coagulation factor Xa, particularly as therapeutically active substances for the treatment and/or prophylaxis of thrombotic disorders, arterial thrombosis, venous thrombosis, deep vein thrombosis, peripheral arterial occlusive disease, unstable angina pectoris, myocardial infarction, coronary artery disease, pulmonary embolism, stroke due to atrial fibrillation, inflammation, arteriosclerosis, acute vessel closure associated with thrombolytic therapy or restenosis, and/or tumour.

In another preferred embodiment, the invention relates to a method for the therapeutic and/or prophylactic treatment of diseases which are associated with the coagulation factor Xa, particularly for the therapeutic and/or prophylactic treatment of thrombotic disorders, arterial thrombosis, venous thrombosis, deep vein thrombosis, peripheral arterial occlusive disease, unstable angina pectoris, myocardial infarction, coronary artery disease, pulmonary embolism, stroke due to atrial fibrillation, inflammation, arteriosclerosis, acute vessel closure associated with thrombolytic therapy or restenosis, and/or tumour, which method comprises administering a compound as defined above to a human being or animal.

The invention also embraces the use of compounds as defined above for the therapeutic and/or prophylactic treatment of diseases which are associated with the coagulation factor Xa, particularly for the therapeutic and/or prophylactic treatment of thrombotic disorders, arterial thrombosis, venous thrombosis, deep vein thrombosis, peripheral arterial occlusive disease, unstable angina pectoris, myocardial infarction, coronary artery disease, pulmonary embolism, stroke due to atrial fibrillation, inflammation, arteriosclerosis, acute vessel closure associated with thrombolytic therapy or restenosis, and/or tumour.

The invention also relates to the use of compounds as described above for the preparation of medicaments for the therapeutic and/or prophylactic treatment of diseases which are associated with the coagulation factor Xa, particularly for the therapeutic and/or prophylactic treatment of thrombotic disorders, arterial thrombosis, venous thrombosis, deep vein thrombosis, peripheral arterial occlusive disease, unstable angina pectoris, myocardial infarction, coronary artery disease, pulmonary embolism, stroke due to atrial fibrillation, inflammation, arteriosclerosis, acute vessel closure associated with thrombolytic therapy or restenosis, and/or tumour. Such medicaments comprise a compound as described above.

The invention also relates to the process and the intermediates for manufacturing the compounds of formula (I) as well as the process for manufacturing the intermediates.

The inhibition of the coagulation factor Xa by the compounds of the present invention can be demonstrated with the aid of a chromogenic peptide substrate assay as described hereinafter.

The activity of the low molecular weight substances can, moreover, be characterized in the “prothrombin time” (PT) clotting test. The substances are prepared as a 10 mM solution in DMSO and thereafter are made up to the desired dilution in the same solvent. Thereafter, 0.25 ml of human plasma (obtained from whole blood anticoagulated with 1/10 volume of 108 mM Na citrate) is placed in an instrument-specific sample container. In each case 5 μl of each dilution of the substance-dilution series is then mixed with the plasma provided. This plasma/inhibitor mixture is incubated at 37° C. for 2 minutes. Thereafter, there is pipetted to the semi-automatic device (ACL, Automated Coagulation Laboratory (Instrument Laboratory)) 50 μl of plasma/inhibitor mixture in the measurement container. The clotting reaction is initiated by the addition of 0.1 ml of Dade® Innovin® (recombinant human tissue factor combined with calcium buffer and synthetic phospholipids, Dade Behring, Inc., Cat. B4212-50). The time up to the fibrin cross-linking is determined photooptically from the ACL. The inhibitor concentration, which brought about a doubling of the PT clotting time, is determined by fitting the data to an exponential regression (XLfit).

The compounds of the present invention can furthermore be characterised by the Activated Partial Thromboplastin time (aPTT). This coagulation test can e.g. be run on the ACL 300 Coagulation System (Instrumentation Laboratory) automatic analyzer. The substances are prepared as a 10 mM solution in DMSO and thereafter made up to the desired dilution in the same solvent. The test is performed with the Dade® Actin® FS Activated PTT reagent (purified soy phosphatides in 1.0×10⁻⁴M ellagic acid, stabilizers and preservative, Dade Behring, Inc., Cat. B4218-100). Thereafter, 0.25 ml aliquots of human plasma (obtained from whole blood anticoagulated with 1/10 volume of 108 mM Na citrate) are spiked with 5 μl of test compound in at least 6 concentrations. 50 μl plasma at 4° C. containing 1/50 vol. inhibitor in solvent are incubated with 50 μl Dade® Actin® FS Activated PTT reagent in water at 37° C. for 3 min., then 50 μl CaCl₂.2H₂O 25 mM in water at 37° C. are added. The time up to the fibrin cross-linking is determined photooptically from the ACL. The inhibitor concentration, which brought about a doubling of the APTT clotting time, is determined by fitting the data to an exponential regression (XLfit).

The Kᵢ values of the active compounds of the present invention preferably amount to about 0.001 to 50 μM, especially about 0.001 to 1 μM. The PT values preferably amount to about 0.5 to 100 μM, especially to about 0.5 to 10 μM. The aPTT values preferably amount to about 0.5 to 100 μM, especially to about 0.5 to 10 μM.

The compounds of formula (I) and/or their pharmaceutically acceptable salts can be used as medicaments, e.g. in the form of pharmaceutical preparations for enteral, parenteral or topical administration. They can be administered, for example, perorally, e.g. in the form of tablets, coated tablets, dragées, hard and soft gelatine capsules, solutions, emulsions or suspensions, rectally, e.g. in the form of suppositories, parenterally, e.g. in the form of injection solutions or suspensions or infusion solutions, or topically, e.g. in the form of ointments, creams or oils. Oral administration is preferred.

The production of the pharmaceutical preparations can be effected in a manner which will be familiar to any person skilled in the art by bringing the described compounds of formula I and/or their pharmaceutically acceptable salts, optionally in combination with other therapeutically valuable substances, into a galenical administration form together with suitable, non-toxic, inert, therapeutically compatible solid or liquid carrier materials and, if desired, usual pharmaceutical adjuvants.

Suitable carrier materials are not only inorganic carrier materials, but also organic carrier materials. Thus, for example, lactose, corn starch or derivatives thereof, talc, stearic acid or its salts can be used as carrier materials for tablets, coated tablets, dragées and hard gelatine capsules. Suitable carrier materials for soft gelatine capsules are, for example, vegetable oils, waxes, fats and semi-solid and liquid polyols (depending on the nature of the active ingredient no carriers might, however, be required in the case of soft gelatine capsules). Suitable carrier materials for the production of solutions and syrups are, for example, water, polyols, sucrose, invert sugar. Suitable carrier materials for injection solutions are, for example, water, alcohols, polyols, glycerol and vegetable oils. Suitable carrier materials for suppositories are, for example, natural or hardened oils, waxes, fats and semi-liquid or liquid polyols. Suitable carrier materials for topical preparations are glycerides, semi-synthetic and synthetic glycerides, hydrogenated oils, liquid waxes, liquid paraffins, liquid fatty alcohols, sterols, polyethylene glycols and cellulose derivatives.

Usual stabilizers, preservatives, wetting and emulsifying agents, consistency-improving agents, flavour-improving agents, salts for varying the osmotic pressure, buffer substances, solubilizers, colorants and masking agents and antioxidants come into consideration as pharmaceutical adjuvants.

The dosage of the compounds of formula (I) can vary within wide limits depending on the disease to be controlled, the age and the individual condition of the patient and the mode of administration, and will, of course, be fitted to the individual requirements in each particular case. For adult patients a daily dosage of about 1 to 1000 mg, especially about 1 to 300 mg, comes into consideration. Depending on severity of the disease and the precise pharmacokinetic profile the compound could be administered with one or several daily dosage units, e.g. in 1 to 3 dosage units.

The pharmaceutical preparations conveniently contain about 1-500 mg, preferably 1-100 mg, of a compound of formula (I).

The following Examples serve to illustrate the present invention in more detail. They are, however, not intended to limit its scope in any manner.

### EXAMPLES

### Example AA

Factor Xa activity was measured spectrophotometrically in microtiter plates in a final volume of 150 μl using the following conditions: Inhibition of human factor Xa (Enzyme Research Laboratories) was tested at an enzyme concentration of 3 nM using the chromogenic substrate S-2222 (Chromogenix AB, Mölndal, Sweden) at 200 nM. The reaction kinetics of the enzyme and the substrate were linear with both time and the enzyme concentration. The inhibitors were dissolved in DMSO and tested at various concentrations up to 100 μM. The inhibitors were diluted using HNPT buffer consisting of HEPES 100 mM, NaCl 140 mM, PEG 6000 0.1% and Tween 80 0.02%, pH 7.8. The cleavage of S-2222 by human factor Xa was followed at 405 nm for 5 minutes at room temperature. The velocity of the reaction was determined by the autoreader from the slope of the linear regression fit to 7 time points (1 minute). The initial velocity for each inhibitor concentration was determined by the slope of at least 4 time points in the linear phase by a linear regression fit (mOD/min²). Apparent dissociation constants Kᵢ were calculated according to Cheng and Prusoff [Cheng, Y. C.; Prusoff, W. H. Relationship between the inhibition constant (Kᵢ) and the concentration of the inhibitor that causes 50 percent inhibition (IC₅₀) of an enzyme reaction. Biochem. Pharmacol. 1973, 22, 3099-3108.] based on the IC₅₀ and the respective Kₘ, determined previously (Kᵢ=IC₅₀/(1+S/Kₘ)). The Kₘ for the substrate used was determined under the conditions of the test with at least 5 substrate concentrations ranging from 0.5 to 15 times Kₘ. [Lottenberg R, Hall J A, Blinder M, Binder E P, Jackson C M., The action of thrombin on peptide p-nitroanilide substrates. Substrate selectivity and examination of hydrolysis under different reaction conditions. Biochim Biophys Acta. 1983 Feb. 15; 742(3):539-57]. According to Eadie [Eadie G. S. The inhibition of cholinesterase by physostigmine and prostigmine. J. Biol. Chem. 1942, 146, 85-93.], the Kₘ for S-2222 amounted to 613 μM.

| | Kᵢ [nM] |
|---|---|
| Example | factor Xa |
| Example 4 | 2 |
| Example 25 | 2 |
| Example 44 | 2 |

### Example 1

### 1,3-Dihydro-isoindole-1,2-dicarboxylic acid 2-[(4-chloro-phenyl)-amide]1-{[2-fluoro-4-(2-oxo-2H-pyridin-1-yl)-phenyl]-amide}

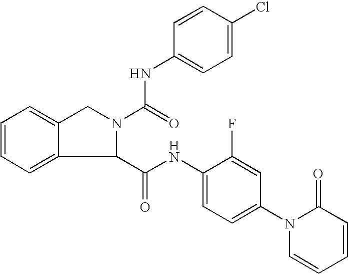

### A 1-[2-Fluoro-4-(2-oxo-2H-pyridin-1-yl)-phenylcarbamoyl]-1,3-dihydro-isoindole-2-carboxylic acid tert-butyl ester

To a solution of 2,3-dihydro-isoindole-1,2-dicarboxylic acid 2-tert-butyl ester (344 mg, CAS 221352-46-1), 1-(4-amino-3-fluoro-phenyl)-1H-pyridin-2-one (265 mg; CAS 536747-52-1) and DIPEA (0.34 ml) in 10 ml acetonitrile and 1 ml DMF was added BOP-Cl (382 mg). The reaction mixture was stirred for 24 hrs at rt, diluted with AcOEt and washed with 1M HCl, 1M NaOH and brine. The organic layers were dried over magnesium sulfate, evaporated and purified by chromatography (silica gel; AcOEt) to deliver the title compound as a yellow oil (280 mg). MS: 450.4 (M+H)⁺.

### B 2,3-Dihydro-1H-isoindole-1-carboxylic acid [2-fluoro-4-(2-oxo-2H-pyridin-1-yl)-phenyl]-amide

A solution of 1-[2-fluoro-4-(2-oxo-2H-pyridin-1-yl)-phenylcarbamoyl]-1,3-dihydro-isoindole-2-carboxylic acid tert-butyl ester (280 mg) in 2 ml 4M HCl/dioxane was stirred 18 h at rt. The reaction mixture was portioned between AcOEt and 1M NaOH/ice. The organic layers were washed with brine, dried over magnesium sulfate and evaporated to deliver a white residue (130 mg) of the title compound. MS: 350.5 (M+H)⁺.

### C 1,3-Dihydro-isoindole-1,2-dicarboxylic acid 2-[(4-chloro-phenyl)-amide]1-{[2-fluoro-4-(2-oxo-2H-pyridin-1-yl)-phenyl]-amide}

To a solution of 2,3-dihydro-1H-isoindole-1-carboxylic acid [2-fluoro-4-(2-oxo-2H-pyridin-1-yl)-phenyl]-amide (130 mg) in 5 ml dichloromethane at 0° C., 4-chlorophenyl-isocyanate (58 mg) was added. The reaction mixture was kept for 1 hr under ice cooling, then heptane was added and the precipitate filtrated. 1,3-Dihydro-isoindole-1,2-dicarboxylic acid 2-[(4-chloro-phenyl)-amide]1-{[2-fluoro-4-(2-oxo-2H-pyridin-1-yl)-phenyl]-amide was obtained as a white solid (104 mg). MS: 503.1 (M+H)⁺.

### Example 2

### 1,3-Dihydro-isoindole-1,2-dicarboxylic acid 2-[(5-chloro-pyridin-2-yl)-amide]1-{[2-fluoro-4-(2-oxo-2H-pyridin-1-yl)-phenyl]-amide}

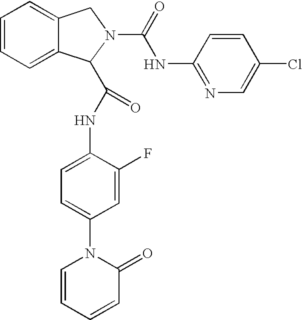

A solution of 2,3-dihydro-1H-isoindole-1-carboxylic acid [2-fluoro-4-(2-oxo-2H-pyridin-1-yl)-phenyl]-amide (example 1B), 90 mg), (5-chloro-pyridin-2-yl)-carbamic acid 4-nitro-phenyl ester (83 mg; CAS 536746-34-6) and DIPEA (0.18 ml) in 5 ml DMF was heated for 3 hrs at 90° C. The reaction mixture was cooled, diluted with AcOEt, washed twofold with 1M NaOH, 1M HCl and brine. The aqueous layers were extracted with AcOEt, dried over magnesium sulfate, evaporated and purified by chromatography (silica gel, AcOEt) to yield the title compound as a white solid (74 mg). MS: 504.4 (M+H)⁺.

### Example 3

### (R)-1,3-Dihydro-isoindole-1,2-dicarboxylic acid 2-[(4-chloro-phenyl)-amide]1-{[4-(2-oxo-2H-pyridin-1-yl)-phenyl]-amide}

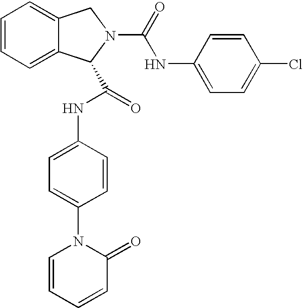

In analogy to example 1, starting from rac-1,3-dihydro-isoindole-1,2-dicarboxylic acid 2-tert-butyl ester (CAS 221352-46-1) and 1-(4-amino-phenyl)-1H-pyridin-2-one (CAS 4444002-64-6) and using a chiral separation (HPLC Chiralcel OD; ethanol/heptane) in the second step, (R)-1,3-dihydro-isoindole-1,2-dicarboxylic acid 2-[(4-chloro-phenyl)-amide]1-{[4-(2-oxo-2H-pyridin-1-yl)-phenyl]-amide} was obtained as a white solid (17 mg). MS: 485.2 (M+H)⁺.

### Example 4

### (S)-1,3-Dihydro-isoindole-1,2-dicarboxylic acid 2-[(4-chloro-phenyl)-amide]1-{[4-(2-oxo-2H-pyridin-1-yl)-phenyl]-amide}

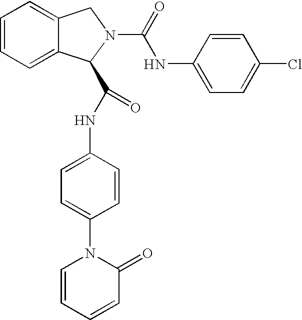

In analogy to example 1, starting from rac-1,3-dihydro-isoindole-1,2-dicarboxylic acid 2-tert-butyl ester (CAS 221352-46-1) and 1-(4-amino-phenyl)-1H-pyridin-2-one (CAS 4444002-64-6) and using a chiral separation (HPLC Chiralcel OD; ethanol/heptane) in the second step, (S)-1,3-dihydro-isoindole-1,2-dicarboxylic acid 2-[(4-chloro-phenyl)-amide]1-{[4-(2-oxo-2H-pyridin-1-yl)-phenyl]-amide} was obtained as a white solid (24 mg). MS: 485.2 (M+H)⁺.

### Example 5

### (R)-1,3-Dihydro-isoindole-1,2-dicarboxylic acid 2-[(5-chloro-pyridin-2-yl)-amide]1-{[4-(2-oxo-2H-pyridin-1-yl)-phenyl]-amide}

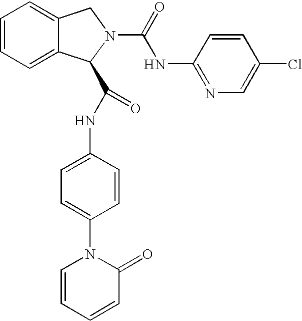

Using a similar procedure described in Example 2, starting from 1,3-dihydro-isoindole-1,2-dicarboxylic acid 2-tert-butyl ester (CAS 221352-46-1) and a chiral HPLC (Chiralcel OD; ethanol/heptane) in the second step, the title compound was obtained as a white solid (34 mg). MS: 486.2 (M+H)⁺.

### Example 6

### (S)-1,3-Dihydro-isoindole-1,2-dicarboxylic acid 2-[(5-chloro-pyridin-2-yl)-amide]1-{[4-(2-oxo-2H-pyridin-1-yl)-phenyl]-amide}

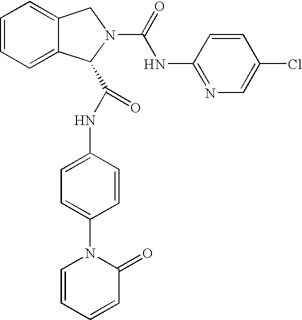

Using a similar procedure described in example 2, starting from 1,3-dihydro-isoindole-1,2-dicarboxylic acid 2-tert-butyl ester (CAS 221352-46-1) and a chiral HPLC (Chiralcel OD; ethanol/heptane) in the second step, the title compound was obtained as a white solid (35 mg). MS: 486.2 (M+H)⁺.

### Example 7

### (S)-1,3-Dihydro-isoindole-1,2-dicarboxylic acid 2-[(5-chloro-pyridin-2-yl)-amide]1-{[4-(2-oxo-2H-pyridin-1-yl)-phenyl]-amide}

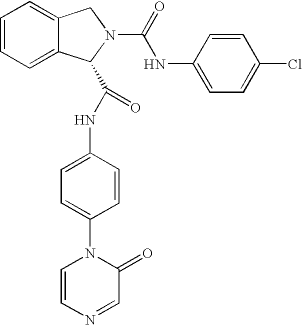

Using a similar procedure described in example 1, starting from 1,3-dihydro-isoindole-1,2-dicarboxylic acid 2-tert-butyl ester (CAS 221352-46-1) and 1-(4-amino-phenyl)-1H-pyrazin-2-one (CAS 4444002-64-6), and after a chiral HPLC (Chiralcel OD; ethanol/heptane) in the second step, the title compound was obtained as a white solid (21 mg). MS: 486.3 (M+H)⁺.

### Example 8

### (R)-1,3-Dihydro-isoindole-1,2-dicarboxylic acid 2-[(5-chloro-pyridin-2-yl)-amide]1-{[4-(2-oxo-2H-pyridin-1-yl)-phenyl]-amide}

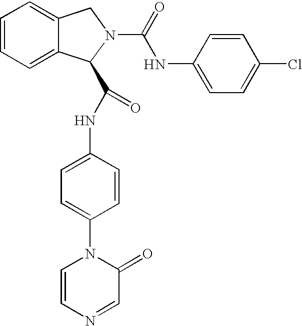

Using a similar procedure described in example 1, starting from 1,3-dihydro-isoindole-1,2-dicarboxylic acid 2-tert-butyl ester (CAS 221352-46-1) and 1-(4-amino-phenyl)-1H-pyrazin-2-one (CAS 4444002-64-6), and after a chiral HPLC (Chiralcel OD; ethanol/heptane) in the second step, the title compound was obtained as a white solid (13 mg). MS: 486.3 (M+H)⁺.

### Example 9

### 1,3-Dihydro-isoindole-1,2-dicarboxylic acid 2-[(4-chloro-phenyl)-amide]1-{[2-fluoro-4-(2-oxo-2H-pyrazin-1-yl)-phenyl]-amide}

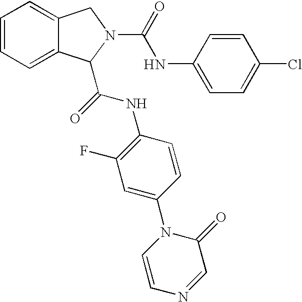

Using a similar procedure described in example 1, starting from 1,3-dihydro-isoindole-1,2-dicarboxylic acid 2-tert-butyl ester (CAS 221352-46-1) and 1-(4-amino-3-fluorophenyl)-1H-pyrazin-2-one, the title compound was obtained as a white solid (21 mg). MS: 504.3 (M+H)⁺.

### Example 10

### 1,3-Dihydro-isoindole-1,2-dicarboxylic acid 2-[(5-chloro-pyridin-2-yl)-amide]1-{[2-fluoro-4-(2-oxo-2H-pyrazin-1-yl)-phenyl]-amide}

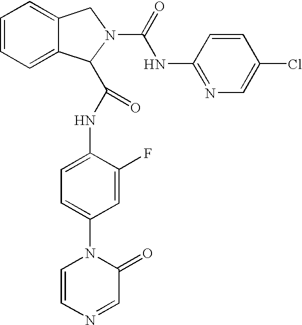

Using a similar procedure described in Example 2, starting from 1,3-dihydro-isoindole-1,2-dicarboxylic acid 2-tert-butyl ester (CAS 221352-46-1) and 1-(4-amino-3-fluorophenyl)-1H-pyrazin-2-one, the title compound was obtained as a white solid (50 mg). MS: 505.1 (M+H)⁺.

### Example 11

### 1,3-Dihydro-isoindole-1,2-dicarboxylic acid 2-[(4-chloro-phenyl)-amide]1-{[4-(2-dimethylaminomethyl-imidazol-1-yl)-2-fluoro-phenyl]-amide}

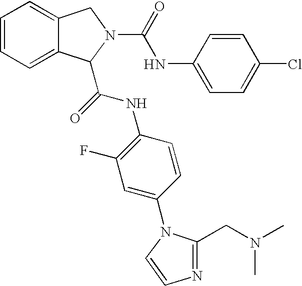

Using a similar procedure described in example 1, starting from 1,3-dihydro-isoindole-1,2-dicarboxylic acid 2-tert-butyl ester (CAS 221352-46-1) and 4-(2-dimethylaminomethyl-imidazol-1-yl)-2-fluoro-phenylamine (CAS 218301-68-9), the title compound was obtained as a white solid (73 mg). MS: 533.5 (M+H)⁺.

### Example 12

### 1,3-Dihydro-isoindole-1,2-dicarboxylic acid 2-[(4-chloro-phenyl)-amide]1-{[2-fluoro-4-(3-oxo-morpholin-4-yl)-phenyl]-amide}

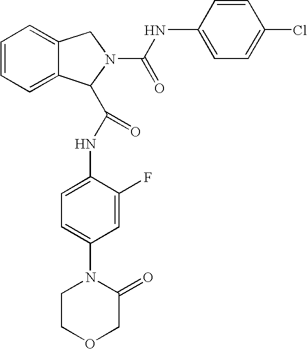

Using a similar procedure described in example 1, starting from 1,3-dihydro-isoindole-1,2-dicarboxylic acid 2-tert-butyl ester (CAS 221352-46-1) and 4-(4-amino-3-fluoro-phenyl)-morpholin-3-one (CAS 742073-22-9), the title compound was obtained as a white solid (54 mg). MS: 526.3 (M+NH₄)⁺.

### Example 13

### 1,3-Dihydro-isoindole-1,2-dicarboxylic acid 2-[(5-chloro-pyridin-2-yl)-amide]1-{[2-fluoro-4-(3-oxo-morpholin-4-yl)-phenyl]-amide}

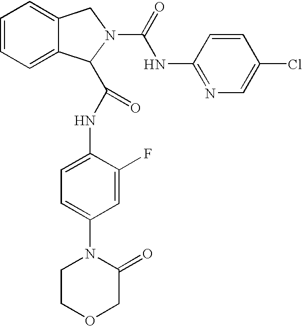

Using a similar procedure described in Example 2, starting from 1,3-dihydro-isoindole-1,2-dicarboxylic acid 2-tert-butyl ester (CAS 221352-46-1) and 4-(4-amino-3-fluoro-phenyl)-morpholin-3-one (CAS 742073-22-9), the title compound was obtained as a white solid (86 mg). MS: 510.4 (M+H)⁺.

### Example 14

### N²-(4-chlorophenyl)-N-[4-(1,1-dioxido-1,2-thiazinan-2-yl)phenyl]-1,3-dihydro-2H-isoindole-1,2-dicarboxamide

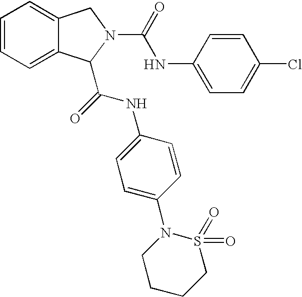

Using a similar procedure described in example 1, starting from 1,3-dihydro-isoindole-1,2-dicarboxylic acid 2-tert-butyl ester (CAS 221352-46-1) and 4-(1,1-dioxido-1,2-thiazinan-2-yl)aniline (CAS 37441-49-9), the title compound was obtained as a white solid (45 mg). MS: 525.5 (M+H)⁺.

### Example 15

### N²-(4-chlorophenyl)-N′-[4-(1,1-dioxido-1,2-thiazinan-2-yl)-2-fluorophenyl]-1,3-dihydro-2H-isoindole-1,2-dicarboxamide

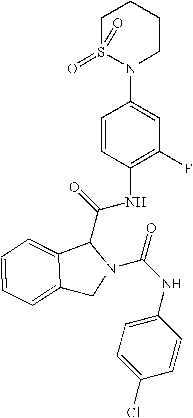

Using a similar procedure described in example 1, starting from 1,3-dihydro-isoindole-1,2-dicarboxylic acid 2-tert-butyl ester (CAS 221352-46-1) and 4-(1,1-dioxido-1,2-thiazinan-2-yl)-2-fluoroaniline (prepared from 2H-1,2-thiazine, tetrahydro-, 1,1-dioxide, CAS 37441-50-2 by reaction with 4-bromo-2-fluoroaniline, K₂CO₃ and CuI in dioxane at 120° C.), the title compound was obtained as a white solid (120 mg). MS: 543.3 (M+H)⁺.

### Example 16

### N²-(5-chloropyridin-2-yl)-N¹-[4-(1,1-dioxido-1,2-thiazinan-2-yl)-2-fluorophenyl]-1,3-dihydro-2H-isoindole-1,2-dicarboxamide

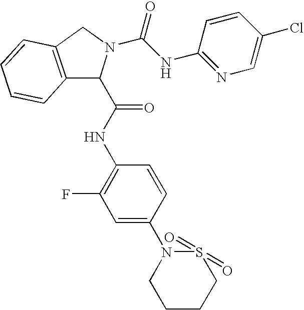

Using a similar procedure described in Example 2, starting from 1,3-dihydro-isoindole-1,2-dicarboxylic acid 2-tert-butyl ester (CAS 221352-46-1) and 4-(1,1-dioxido-1,2-thiazinan-2-yl)-2-fluoroaniline (prepared by reaction of 2H-1,2-thiazine, tetrahydro-, 1,1-dioxide, CAS 37441-50-2 with 4-bromo-2-fluoroaniline, K₂CO₃ and CuI in dioxane at 120° C.), the title compound was obtained as a white solid (104 mg). MS: 544.2 (M+H)⁺.

### Example 17

### 1-(4-Pyridin-4-yl-piperazine-1-carbonyl)-1,3-dihydro-isoindole-2-carboxylic acid (4-chloro-phenyl)-amide

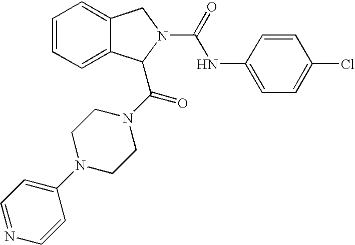

Using a similar procedure described in example 1, starting from 1,3-dihydro-isoindole-1,2-dicarboxylic acid 2-tert-butyl ester (CAS 221352-46-1) and 1-(4-pyridinyl)piperazine (CAS 1008-91-9), the title compound was obtained as a white solid (35 mg). MS: 462.0 (M+H)⁺.

### Example 18

### 1-Methyl-1,3-dihydro-isoindole-1,2-dicarboxylic acid 2-[(4-chloro-phenyl)-amide]1-{[2-fluoro-4-(2-oxo-2H-pyridin-1-yl)-phenyl]-amide}

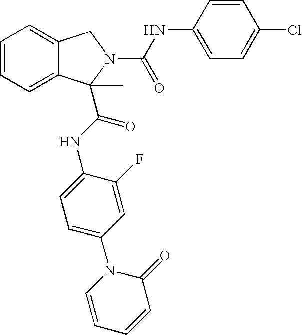

### A 1-Methyl-1,3-dihydro-isoindole-1,2-dicarboxylic acid 2-benzyl ester

A solution of 1-methyl-1,3-dihydro-isoindole-1,2-dicarboxylic acid 2-benzyl ester 1-tert-butyl ester (CAS 401504-28-7; 1.65 g) in 8 ml dichloromethane, 0.49 ml anisole and 10 ml trifluoroacetic acid was stirred 4.5 hrs at 0° C. The reaction mixture was poured onto 1M NaOH/ice. The basic aqueous phase was washed with dichloromethane, and then acidified to pH 2, and the product extracted with three portions of dichloromethane. The organic layers were dried over magnesium suphate, evaporated and taken without purification for the next step (1.28 g). MS: 310.4 (M−H)¹.

### B 1-Methyl-2,3-dihydro-1H-isoindole-1-carboxylic acid

A suspension of 1-methyl-1,3-dihydro-isoindole-1,2-dicarboxylic acid 2-benzyl ester (1.2 g) and Pd/C 10% (120 mg) in 20 ml was vigorously stirred 3 hrs at rt under an hydrogene atmosphere. The reaction mixture was filtered, evaporated and the product precipitated with AcOEt. The title compound was delivered as a white solid (534 mg). MS: 176.2 (M−H)¹.

### C 1-Methyl-1,3-dihydro-isoindole-1,2-dicarboxylic acid 2-tert-butyl ester

To a solution of 1-methyl-2,3-dihydro-1H-isoindole-1-carboxylic acid (469 mg) in 18 ml acetonitrile and 1.5 ml water, were added successively triethylamine (0.92 ml), DMAP (6 mg) and Boc₂O (866 mg). After 3 hrs at rt, the reaction mixture was treated with 1M NaOH, the aqueous layers washed with dichloromethane, then acidified to pH2 and extracted with dichloromethane. The organic layers were dried over magnesium sulfate and evaporated to yield a white residue of the title compound (712 mg). MS: 276.2 (M−H)¹.

### D 1-Methyl-1,3-dihydro-isoindole-1,2-dicarboxylic acid 2-[(4-chloro-phenyl)-amide]1-{[2-fluoro-4-(2-oxo-2H-pyridin-1-yl)-phenyl]-amide}

Starting from 1-methyl-1,3-dihydro-isoindole-1,2-dicarboxylic acid 2-tert-butyl ester and using the procedure described in example 1, the title compound was delivered as a white solid (87 mg). MS: 517.2 (M+H)⁺.

### Example 19

### (R)-11-Methyl-1,3-dihydro-isoindole-1,2-dicarboxylic acid 2-[(4-chloro-phenyl)-amide]1-{[2-fluoro-4-(2-oxo-2H-pyridin-1-yl)-phenyl]-amide}

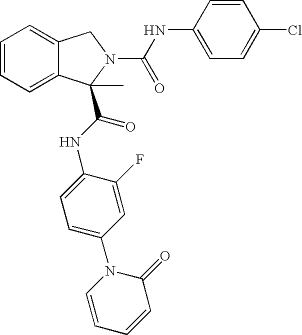

This compound was prepared in analogy to example 18, but using a chiral separation (HPLC Chiralcel OD) of 1-methyl-2,3-dihydro-1H-isoindole-1-carboxylic acid [2-fluoro-4-(2-oxo-2H-pyridin-1-yl)-phenyl]-amide to obtain a white solid (42 mg). MS: 517.2 (M+H)⁺.

### Example 20

### (S)-1-Methyl-1,3-dihydro-isoindole-1,2-dicarboxylic acid 2-[(4-chloro-phenyl)-amide]1-{[2-fluoro-4-(2-oxo-2H-pyridin-1-yl)-phenyl]-amide}

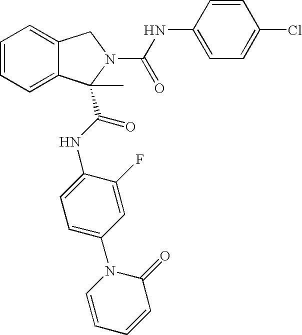

This compound was prepared in analogy to example 18, but using a chiral separation (HPLC Chiralcel OD) of 1-methyl-2,3-dihydro-1H-isoindole-1-carboxylic acid [2-fluoro-4-(2-oxo-2H-pyridin-1-yl)-phenyl]-amide to obtain a white solid (35 mg). MS: 517.2 (M+H)⁺.

### Example 21

### 2-Formyl-2,3-dihydro-1H-isoindole-1,6-dicarboxylic acid 6-dimethylamide 1-{[2-fluoro-4-(2-oxo-2H-pyridin-1-yl)-phenyl]-amide}

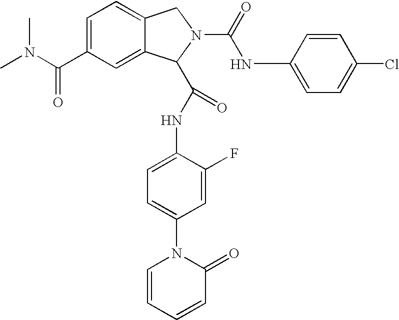

### A 4-[(Benzyl-tert-butoxycarbonylmethyl-amino)-methyl]-3-bromo-benzoic acid methyl ester

To a suspension of tris(dibenzylideneacetone)dipalladium (41 mg), 3,2-(dicyclohexylphosphino)biphenyl (16 mg) and tri-kaliumphosphate (680 mg) in 6 ml DME under argon, were added 3-bromo-4-bromomethyl-benzoic acid methyl ester (700 mg; CAS 78946-25-5; Journal of the American Chemical Society, 124(50), 14993-15000; 2002) and benzylamino-acetic acid tert-butyl ester (603 mg; CAS 7662-76-2). After stirring 2 hrs at 100° C., the suspension was diluted with 80 ml tBuOMe/AcOEt 1/1 and filtrated. The filtrate was evaporated to dryness, the residue diluted with AcOEt and washed with 1M HCl, 1M NaOH and brine. The organic layers were dried over magnesium sulfate, evaporated and chromatographed (silica gel, AcOEt/heptane, 1/3) to deliver the title compound as a colorless oil (510 mg). MS: 448.2/450.2 (M+H)⁺.

### B 4-[(Benzyl-tert-butoxycarbonylmethyl-amino)-methyl]-3-bromo-benzoic acid

A solution of 4-[(benzyl-tert-butoxycarbonylmethyl-amino)-methyl]-3-bromo-benzoic acid methyl ester (300 mg) and lithium hydroxide (48 mg) in 4 ml THF, 1 ml MeOH and 1 ml water was stirred 1 hr at rt. The reaction mixture was diluted with AcOEt and washed with tampon phosphates pH 4 and brine. The organic layers were dried over magnesium sulfate and evaporated to give 4-[(benzyl-tert-butoxycarbonylmethyl-amino)-methyl}-3-bromo-benzoic acid as a colorless oil (295 mg). MS: 434.3/436.1 (M+H)⁺.

### C [Benzyl-(2-bromo-4-dimethylcarbamoyl-benzyl)-amino]-acetic acid tert-butyl ester

A solution of 4-[(benzyl-tert-butoxycarbonylmethyl-amino)-methyl]-3-bromo-benzoic acid (2.7 g), EDCI (1.79 g), HOBT (1.26 g), DIPEA (2.13 ml) and 2M dimethylamine in THF (9.3 ml) in 45 ml acetonitrile was stirred 18 hrs at rt. The reaction mixture was diluted with AcOEt and washed with 0.1 M HCl, 1M NaOH and brine. The organic layers were dried over magnesium sulfate, evaporated and chromatographed to give [benzyl-(2-bromo-4-dimethylcarbamoyl-benzyl)-amino]-acetic acid tert-butyl ester as a light yellow oil (1.64 g). MS: 405.1/407.2 (M+H)⁺.

### D 2-Benzyl-6-dimethylcarbamoyl-2,3-dihydro-1H-isoindole-1-carboxylic acid tert-butyl ester

To a suspension of tris(dibenzylideneacetone)dipalladium (7.5 mg), 2-(dicyclohexylphosphino)-2-(N,N-dimethylamino)-biphenyl (8.1 mg) and lithium tert-butylate (66 mg) in 1 ml dioxane under argon at 85° C., was added a solution of [benzyl-(2-bromo-4-dimethylcarbamoyl-benzyl)-amino]-acetic acid tert-butyl ester (190 mg) in 2 ml dioxane. The reaction mixture was heated 2 hrs at 85° C., evaporated and chromatographed (silica gel, AcOEt/heptane, 3/1) to yield the title compound as a colorless oil (84 mg). MS: 381.5 (M+H)⁺.

### E 2-Benzyl-6-dimethylcarbamoyl-2,3-dihydro-1H-isoindole-1-carboxylic acid tert-butyl ester

Hydrogenation of 2-benzyl-6-dimethylcarbamoyl-2,3-dihydro-1H-isoindole-1-carboxylic acid tert-butyl ester (820 mg) in 20 ml ethanol and a chromatography (silica gel, AcOEt/heptane, 3/1) delivered 2-benzyl-6-dimethylcarbamoyl-2,3-dihydro-1H-isoindole-1-carboxylic acid tert-butyl ester (374 mg) as a yellow solid (374 mg). MS: 291.1 (M+H)⁺.

### F 6-Dimethylcarbamoyl-2,3-dihydro-1H-isoindole-1-carboxylic acid

A solution of 2-benzyl-6-dimethylcarbamoyl-2,3-dihydro-1H-isoindole-1-carboxylic acid tert-butyl ester (100 mg) in 5 ml dichloromethane and 1 ml TFA was stirred 18 hrs under ice cooling. The reaction mixture was evaporated and the brown residue taken without purification for the next step. MS: 235.1 (M+H)⁺.

### G 6-Dimethylcarbamoyl-1,3-dihydro-isoindole-1,2-dicarboxylic acid 2-tert-butyl ester

To a solution of 6-dimethylcarbamoyl-2,3-dihydro-1H-isoindole-1-carboxylic acid (200 mg) and triethylamine (0.12 ml) in 15 ml dioxane at rt, was added a solution of Boc₂O (223 mg) in 5 ml dioxane. The reaction mixture was stirred 18 hrs at rt, evaporated and the brown residue taken without purification for the next step. MS: 335.3 (M+H)⁺.

### H 2-Formyl-2,3-dihydro-1H-isoindole-1,6-dicarboxylic acid 6-dimethylamide 1-{[2-fluoro-4-(2-oxo-2H-pyridin-1-yl)-phenyl]-amide}

The title compound was synthesized from 6-dimethylcarbamoyl-1,3-dihydro-isoindole-1,2-dicarboxylic acid 2-tert-butyl ester in analogy to example IC, and it was delivered as a light brown solid (16 mg). MS: 574.5 (M+H)⁺.

### Example 22

### (R)-2,3-Dihydro-indole-1,2-dicarboxylicacid 1-[(4-chloro-phenyl)-amide]2-{[2-fluoro-4-(2-oxo-2H-pyridin-1-yl)-phenyl]-amide}

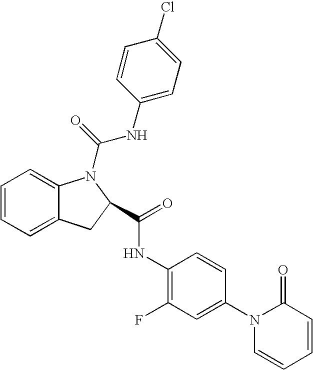

Starting from (R)-2,3-dihydro-indole-1,2-dicarboxylicacid 1-tert-butyl ester (prepared by treatment of (R)-2,3-dihydro-1H-indole-2-carboxylic acid (CAS 98167-06-7) with Boc₂O in dioxane) and using the procedure described in example 1, (R)-2,3-dihydro-indole-1,2-dicarboxylicacid 1-[(4-chloro-phenyl)-amide]2-{[2-fluoro-4-(2-oxo-2H-pyridin-1-yl)-phenyl]-amide} was delivered as a white solid (34 mg). MS: 503.5 (M+H)⁺.

### Example 23

### (R)-2,3-Dihydro-indole-1,2-dicarboxylicacid 1-[(5-chloro-pyridin-2-yl)-amide]2-{[2-fluoro-4-(2-oxo-2H-pyridin-1-yl)-phenyl]-amide}

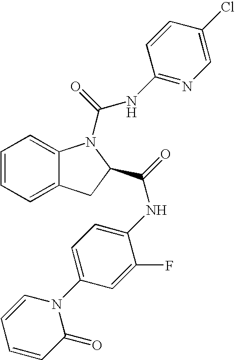

Starting from (R)-2,3-dihydro-indole-1,2-dicarboxylicacid 1-tert-butyl ester (prepared by treatment of (R)-2,3-dihydro-1H-indole-2-carboxylic acid (CAS 98167-06-7) with Boc₂O in dioxane) and using the procedure described in example 2, (R)-2,3-dihydro-indole-1,2-dicarboxylicacid 1-[(5-chloro-pyridin-2-yl)-amide]2-{[2-fluoro-4-(2-oxo-2H-pyridin-1-yl)-phenyl]-amide} was delivered as a white solid (22 mg). MS: 504.4 (M+H)⁺.

### Example 24

### 2,3-Dihydro-indole-1,3-dicarboxylic acid 1-[(4-chloro-phenyl)-amide]3-{[2-fluoro-4-(2-oxo-2H-pyridin-1-yl)-phenyl]-amide}

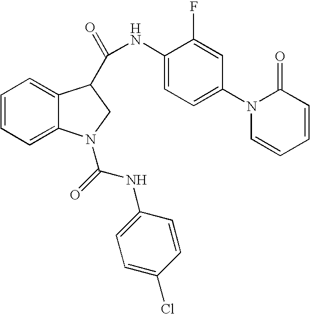

2,3-Dihydro-indole-1,3-dicarboxylic acid 1-tert-butyl ester 3-methyl ester (CAS528862-00-2; Tetrahedron 59(6), 747, 2003) was hydrolysed using the procedure described in example 21B. The title compound was prepared from 2,3-dihydro-indole-1,3-dicarboxylic acid 1-tert-butyl ester (CAS 177201-79-5) in analogy to the method described in example 1 and obtained as a white solid (32 mg). MS: 501.1 (M−H)⁻.

### Example 25

### (R)-3,4-Dihydro-1H-isoquinoline-2,3-dicarboxylic acid 2-[(4-chloro-phenyl)-amide]3-{[2-fluoro-4-(2-oxo-2H-pyridin-1-yl)-phenyl]-amide}

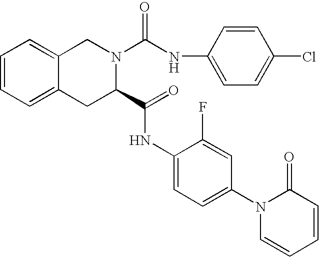

Starting from (R)-3,4-dihydro-1H-isoquinoline-2,3-dicarboxylic acid 2-tert-butyl ester (CAS 115962-35-1) and 1-(4-amino-3-fluoro-phenyl)-1H-pyridin-2-one (CAS 536747-52-1) and using the procedure described in example 1, (R)-3,4-dihydro-1H-isoquinoline-2,3-dicarboxylic acid 2-[(4-chloro-phenyl)-amide]3-{[2-fluoro-4-(2-oxo-2H-pyridin-1-yl)-phenyl]-amide} was obtained as a white solid (112 mg). MS: 517.3 (M+H)⁺.

### Example 26

### (S)-3,4-Dihydro-1H-isoquinoline-2,3-dicarboxylic acid 2-[(4-chloro-phenyl)-amide]3-{[2-fluoro-4-(2-oxo-2H-pyridin-1-yl)-phenyl]-amide}

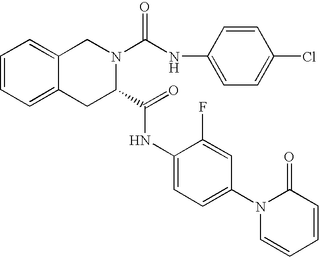

Starting from (S)-3,4-dihydro-1H-isoquinoline-2,3-dicarboxylic acid 2-tert-butyl ester (CAS 78879-20-6) and 1-(4-amino-3-fluoro-phenyl)-1H-pyridin-2-one (CAS 536747-52-1) and using the procedure described in example 1, (S)-3,4-dihydro-1H-isoquinoline-2,3-dicarboxylic acid 2-[(4-chloro-phenyl)-amide]3-{[2-fluoro-4-(2-oxo-2H-pyridin-1-yl)-phenyl]-amide} was obtained as a white solid (24 mg). MS: 517.4 (M+H)⁺.

### Example 27

### (S)-3,4-Dihydro-1H-isoquinoline-2,3-dicarboxylic acid 2-[(4-chloro-phenyl)-amide]3-{[2-fluoro-4-(3-oxo-morpholin-4-yl)-phenyl]-amide}

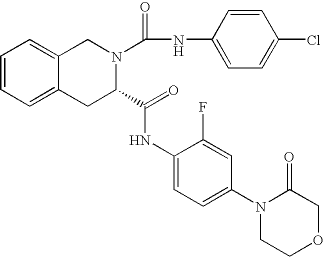

Starting from (S)-3,4-dihydro-1H-isoquinoline-2,3-dicarboxylic acid 2-tert-butyl ester (CAS 78879-20-6) and 4-(4-amino-3-fluoro-phenyl)-morpholin-3-one (CAS 742073-22-9) and using the procedure described in example 1, (S)-3,4-dihydro-1H-isoquinoline-2,3-dicarboxylic acid 2-[(4-chloro-phenyl)-amide]3-{[2-fluoro-4-(3-oxo-morpholin-4-yl)-phenyl]-amide} was obtained as a white solid (51 mg). MS: 523.3 (M+H)⁺.

### Example 28

### (R)-3,4-Dihydro-1H-isoquinoline-2,3-dicarboxylic acid 2-[(4-chloro-phenyl)-amide]3-{[2-fluoro-4-(3-oxo-morpholin-4-yl)-phenyl]-amide}

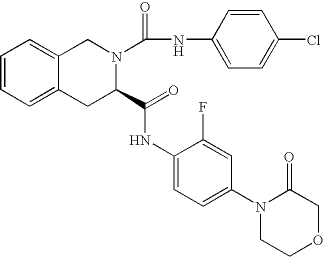

Starting from (R)-3,4-dihydro-1H-isoquinoline-2,3-dicarboxylic acid 2-tert-butyl ester (CAS 115962-35-1) and 4-(4-amino-3-fluoro-phenyl)-morpholin-3-one (CAS 742073-22-9) and using the procedure described in example 1, (R)-3,4-dihydro-1H-isoquinoline-2,3-dicarboxylic acid 2-[(4-chloro-phenyl)-amide]3-{[2-fluoro-4-(3-oxo-morpholin-4-yl)-phenyl]-amide} was obtained as a white solid (71 mg). MS: 523.3 (M+H)⁺.

### Example 29

### (R)-3,4-Dihydro-1H-isoquinoline-2,3-dicarboxylic acid 2-[(4-chloro-phenyl)-amide]3-{[2-fluoro-4-(2-oxo-2H-pyrazin-1-yl)-phenyl]-amide}

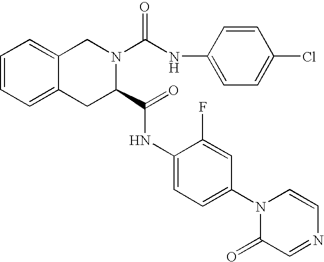

Starting from (R)-3,4-dihydro-1H-isoquinoline-2,3-dicarboxylic acid 2-tert-butyl ester (CAS 115962-35-1) and 1-(4-amino-phenyl)-1H-pyrazin-2-one, (CAS 4444002-64-6) and using the procedure described in example 1, (R)-3,4-dihydro-1H-isoquinoline-2,3-dicarboxylic acid 2-[(4-chloro-phenyl)-amide]3-{[2-fluoro-4-(2-oxo-2H-pyrazin-1-yl)-phenyl]-amide} was obtained as a white solid (5 mg). MS: 518.4 (M+H)⁺.

### Example 30

### (R)-N²-(4-chlorophenyl)-N³-[4-(1,1-dioxido-1,2-thiazinan-2-yl)phenyl]-3,4-dihydroisoquinoline-2,3(1H)-dicarboxamide

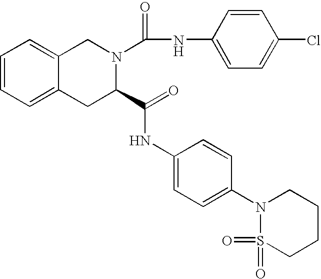

Starting from (R)-3,4-dihydro-1H-isoquinoline-2,3-dicarboxylic acid 2-tert-butyl ester (CAS 115962-35-1) and 4-(1,1-dioxido-1,2-thiazinan-2-yl)aniline (CAS 37441-49-9), and using the procedure described in example 1, the title compound was obtained as white solid (76 mg). MS: 539.5 (M+H)⁺.

### Example 31

### (R)-N²-(4-chlorophenyl)-N³- [4-(1,1-dioxidoisothiazolidin-2-yl)phenyl]-3,4-dihydroisoquinoline-2,3(1H)-dicarboxamide

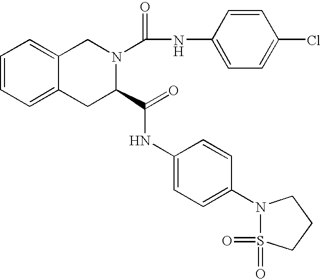

Starting from (R)-3,4-dihydro-1H-isoquinoline-2,3-dicarboxylic acid 2-tert-butyl ester (CAS 115962-35-1) and 4-(1,1-dioxidoisothiazolidin-2-yl)aniline (CAS 90556-91-5) and using the procedure described in example 1, the title compound was obtained as white solid (82 mg). MS: 525.3 (M+H)⁺.

### Example 32

### (R)-3,4-Dihydro-1H-isoquinoline-2,3-dicarboxylic acid 2-[(5-chloro-pyridin-2-yl)-amide]3-{[2-fluoro-4-(2-oxo-2H-pyridin-1-yl)-phenyl]-amide}

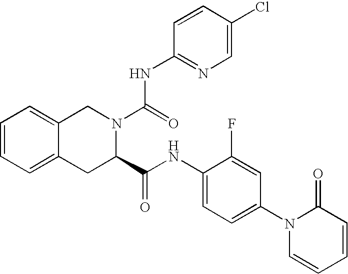

Starting from (R)-3,4-dihydro-1H-isoquinoline-2,3-dicarboxylic acid 2-tert-butyl ester (CAS 115962-35-1) and 1-(4-amino-3-fluoro-phenyl)-1H-pyridin-2-one (CAS 536747-52-1) and using the procedure described in example 2, (R)-3,4-dihydro-1H-isoquinoline-2,3-dicarboxylic acid 2-[(5-chloro-pyridin-2-yl)-amide]3-{[2-fluoro-4-(2-oxo-2H-pyridin-1-yl)-phenyl]-amide} was obtained as a white solid (107 mg). MS: 518.3 (M+H)⁺.

### Example 33

### (R)-3,4-Dihydro-1H-isoquinoline-2,3-dicarboxylic acid 3-{[2-fluoro-4-(2-oxo-2H-pyridin-1-yl)-phenyl]-amide}2-[(4-methoxy-phenyl)-amide]

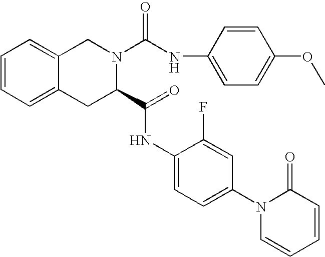

Starting from (R)-3,4-dihydro-1H-isoquinoline-2,3-dicarboxylic acid 2-tert-butyl ester (CAS 115962-35-1) and 1-(4-amino-3-fluoro-phenyl)-1H-pyridin-2-one (CAS 536747-52-1) and using the procedure described in example 1, with 4-methoxyphenyl isocyanate, (R)-3,4-dihydro-1H-isoquinoline-2,3-dicarboxylic acid 3-{[2-fluoro-4-(2-oxo-2H-pyridin-1-yl)-phenyl]-amide}2-[(4-methoxy-phenyl)-amide] was obtained as a white solid (121 mg). MS: 530.2 (M+NH₄)⁺.

### Example 34

### 3,4-Dihydro-1H-isoquinoline-1,2-dicarboxylic acid 2-[(5-chloro-pyridin-2-yl)-amide]1-{[2-fluoro-4-(2-oxo-2H-pyridin-1-yl)-phenyl]-amide}

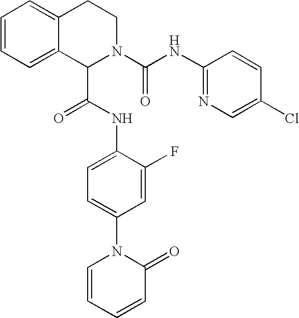

The title compound was synthesized from 3,4-dihydro-1H-isoquinoline-1,2-dicarboxylic acid 2-tert-butyl ester (CAS 166591-85-1; European Journal of medicinal chemistry 36(3), 265, 2001) using the procedure described in example 2 to yield a white solid (73 mg). MS: 518.3 (M+H)⁺.

### Example 35

### 3,4-Dihydro-1H-isoquinoline-2,4-dicarboxylic acid 2-[(4-chloro-phenyl)-amide]4-{[2-fluoro-4-(2-oxo-2H-pyridin-1-yl)-phenyl]-amide}

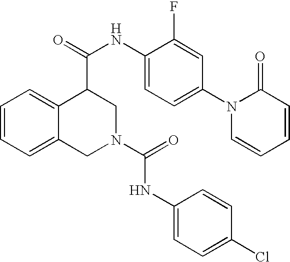

### A 3,4-Dihydro-1H-isoquinoline-2,4-dicarboxylic acid 2-tert-butyl ester 4-methyl ester

To a solution of methyl 1,2,3,4-tetrahydro-isoquinoline-4-carboxylic acid (98 mg; CAS 681448-82-8; Synthetic Communications 34, 137, 2004) in 2 ml dichloromethane under ice cooling, were added successively Boc₂O (223 mg), DIPEA (0.26 ml) and DMAP (6 mg). The reaction mixture was stirred 18 hrs at rt, diluted with AcOEt and washed with 1M HCl, 1M NaOH and brine. The organic layers were dried over magnesium sulfate, evaporated and chromatographed (silica gel, AcOEt/heptane 1/1) to yield the title compound as a yellow oil (135 mg). MS: 292.1 (M+H)⁺.

### B 3,4-Dihydro-1H-isoquinoline-2,4-dicarboxylic acid 2-tert-butyl ester

The above compound (130 mg) was treated with 1M NaOH (1 ml) in 2 ml methanol at rt during 18 hrs. The reaction mixture was washed twice with tBuOMe, the aqueous layer was acidified to pH 3 and extracted with AcOEt. The organic layers were dried over magnesium sulfate and evaporated to deliver a yellow solid (100 mg). MS: 300.0 (M+Na)⁺.

### C 3,4-Dihydro-1H-isoquinoline-2,4-dicarboxylic acid 2-[(4-chloro-phenyl)-amide]4-{[2-fluoro-4-(2-oxo-2H-pyridin-1-yl)-phenyl]-amide}

The title compound was synthesized from 3,4-dihydro-1H-isoquinoline-2,4-dicarboxylic acid 2-tert-butyl ester using the procedure described in example 1 to deliver a white solid (30 mg). MS: 534.3 (M+NH₄)⁺.

### Example 36

### (R)-3,4-Dihydro-1H-isoquinoline-2,3-dicarboxylic acid 3-[(4-chloro-phenyl)-amide]2-{[2-fluoro-4-(2-oxo-2H-pyridin-1-yl)-phenyl]-amide}

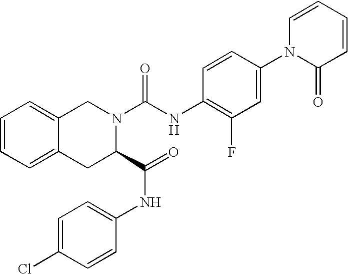

### A (R)-3-(4-Chloro-phenylcarbamoyl)-3,4-dihydro-1H-isoquinoline-2-carboxylic acid tert-butyl ester

To a solution of (R)-3,4-dihydro-1H-isoquinoline-2,3-dicarboxylic acid 2-tert-butyl ester (1 g) in 10 ml DMSO under ice cooling, were added successively HATU (2.74 g), HOBT (0.98 g), 4-chloroaniline (0.54 g) and DIPEA (1.85 ml). After stirring 1 hr at 0° C. and 16 hrs at rt, the reaction mixture was diluted with AcOEt, washed with 10% citric acid solution, 10% NaHCO3 and brine. The organic layers were dried over magnesium sulfate, evaporated and chromatographed (silica gel, AcOEt/heptane, 3/2) to yield a yellow solid (1.36 g). MS: 409.3 (M+Na)⁺.

### B (R)-1,2,3,4-Tetrahydro-isoquinoline-3-carboxylic acid (4-chloro-phenyl)-amide

Starting from (R)-3-(4-chloro-phenylcarbamoyl)-3,4-dihydro-1H-isoquinoline-2-carboxylic acid tert-butyl ester (1.36 g) and using the procedure described in example 1, the title compound was delivered as a yellow solid (0.933 g). MS: 287.1 (M+H)⁺.

### C (R)-3,4-Dihydro-1H-isoquinoline-2,3-dicarboxylic acid 3-[(4-chloro-phenyl)-amide]2-{[2-fluoro-4-(2-oxo-2H-pyridin-1-yl)-phenyl]-amide}

A solution of (R)-1,2,3,4-tetrahydro-isoquinoline-3-carboxylic acid (4-chloro-phenyl)-amide (70 mg), DIPEA (0.06 ml) and [2-fluoro-4-(2-oxo-2H-pyridin-1-yl)-phenyl]carbamic acid-4-nitro-phenyl ester (99 mg; prepared from 1-(4-amino-3-fluoro-phenyl)-1H-pyridin-2-one by reaction with 4-nitrophenyl-chlorformiat and pyridine in dichloromethane) in 2 ml DMF was heated 1 hr at 80° C. The reaction mixture was diluted with AcOEt and washed with 1M NaOH and brine. The organic layers were dried over magnesium sulfate, evaporated and chromatographed (silica gel, AcOEt) to yield the title compound as a white solid (13 mg). MS: 534.3 (M+NH₄)⁺.

### Example 37

### (3R)-Octahydro-isoquinoline-2,3-dicarboxylic acid 2-[(4-chloro-phenyl)-amide]3-{[2-fluoro-4-(2-oxo-2H-pyridin-1-yl)-phenyl]-amide}

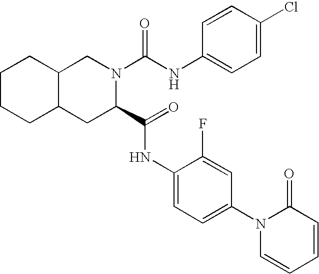

Starting from octahydro-isoquinoline-2,3-dicarboxylic acid 2-tert-butyl ester (CAS 312639-54-6) prepared by hydrogenation of 1,2,3,4-tetrahydro-isoquinoline-3-carboxylic acid and bocylation of the intermediate, and using the procedure described in example 1, (3R)-octahydro-isoquinoline-2,3-dicarboxylic acid 2-[(4-chloro-phenyl)-amide]3-{[2-fluoro-4-(2-oxo-2H-pyridin-1-yl)-phenyl]-amide} was obtained as a light yellow solid (3.6 mg). MS: 525.5 (M+H)⁺.

### Example 38

### 3-{[(5-Chloro-thiophene-2-carbonyl)-amino]-methyl}-2,3-dihydro-indole-1-carboxylic acid [2-fluoro-4-(2-oxo-2H-pyridin-1-yl)-phenyl]-amide

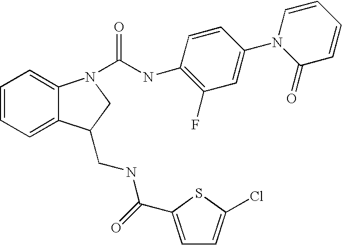

### A (2,3-Dihydro-1H-indol-3-yl)-methanol

To a solution of 2,3-dihydro-indole-1,3-dicarboxylic acid 1-tert-butyl ester 3-methyl ester (740 mg; CAS 528862-00-2; Tetrahedron 59(6), 747, 2003) in 20 ml methanol under ice cooling, natrium borhydride (810 mg) was added. The reaction mixture was stirred at 0° C. for 2 hrs and at ambient temperature under argon for another 3 hrs. The crude reaction mixture was poured on 200 ml NH₄Cl/AcOEt, the phases were separated. The organic phase was washed with 1M NaOH and brine, dried over magnesium sulfate and concentrated. The residue was purified by column chromatography (silica gel, AcOEt/heptane, 2/1) to yield (2,3-dihydro-1H-indol-3-yl)-methanol (562 mg) as a colorless oil. MS: 250.3 (M+H)⁺.

### B Methanesulfonic acid 2,3-dihydro-1H-indol-3-ylmethyl ester

Methanesulfonylchlorid (0.38 ml) was added to a cooled solution of (2,3-dihydro-1H-indol-3-yl)-methanol (540 mg) and DIPEA (0.90 ml) in 5 ml CH₂Cl₂. The reaction mixture was stirred 1 hr at 0° C., washed with 1M HCl/ice and brine. The aqueous phases were extracted with CH₂Cl₂. The organic layers were dried over magnesium sulfate and concentrated. The oily residue of methanesulfonic acid 2,3-dihydro-1H-indol-3-ylmethyl ester (714 mg) was used without purification for the next step. MS: 328.3 (M+H)⁺.

### C 3-Azidomethyl-2,3-dihydro-1H-indole

A solution of methanesulfonic acid 2,3-dihydro-1H-indol-3-ylmethyl ester (710 mg) and NaN₃ (155 mg) in 15 ml DMF was heated 18 hrs at 50° C. The reaction mixture was evaporated and chromatographed (silica gel, AcOEt/heptane, 1/3) to yield 3-azidomethyl-2,3-dihydro-1H-indole (364 mg) as a colorless oil. MS-EI: 274.2 (M).

### D (2,3-Dihydro-1H-indol-3-yl)-methylamine

A suspension of 3-azidomethyl-2,3-dihydro-1H-indole (340 mg) and Pd/C 10% (40 mg) in 8 ml methanol was vigorously stirred at rt under hydrogene atmosphere (10 bar) during 24 hrs. Filtration of the catalyst and evaporation of the solvents delivered a colorless oil of (2,3-dihydro-1H-indol-3-yl)-methylamine (273 mg). MS: 249.4 (M+H)⁺.

### E 3-{[(5-Chloro-thiophene-2-carbonyl)-amino]-methyl}-2,3-dihydro-indole-1-carboxylic acid tert-butyl ester

A solution of (2,3-dihydro-1H-indol-3-yl)-methylamine (350 mg), 5-chloro-2-thiophencarboxylic acid (275 mg), DIPEA (0.48 ml) and BOP (935 mg) in 10 ml THF was stirred 4 hrs at rt. The reaction mixture was diluted with AcOEt, washed with 1M HCl, 1M NaOH and brine. The organic layers were dried over magnesium sulfate, evaporated and chromatographed (silica gel, AcOEt/heptane, 2/3) to yield 3-{[(5-chloro-thiophene-2-carbonyl)-amino]-methyl}-2,3-dihydro-indole-1-carboxylic acid tert-butyl ester as a white solid (475 mg). MS: 393.1 (M)⁺.

### F (3R)5-Chloro-thiophene-2-carboxylic acid (2,3-dihydro-1H-indol-3-ylmethyl)-amide and (3S)-5-Chloro-thiophene-2-carboxylic acid (2,3-dihydro-1H-indol-3-ylmethyl)-amide

To a solution of 3-{[(5-chloro-thiophene-2-carbonyl)-amino]-methyl}-2,3-dihydro-indole-1-carboxylic acid tert-butyl ester (470 mg) in 8 ml dioxane, was added 3 ml 4M HCl/dioxane. The reaction mixture was stirred 18 hrs at rt and extracted with AcOEt. The aqueous layer was basified (pH 9) with NaOH and extracted twice with AcOEt. The organic layers were dried over magnesium sulfate, evaporated and chromatographed (Chiralcel OD; 20% ethanol/heptane) to yield the two enantiomers of 5-chloro-thiophene-2-carboxylic acid (2,3-dihydro-1H-indol-3-ylmethyl)-amide as white solid (97 mg and 93 mg). MS: 292.9 (M)⁺.

### G (3S)3-{[(5-Chloro-thiophene-2-carbonyl)-amino]-methyl}-2,3-dihydro-indole-1-carboxylic acid [2-fluoro-4-(2-oxo-2H-pyridin-1-yl)-phenyl]-amide

A solution of (5R)-5-chloro-thiophene-2-carboxylic acid (2,3-dihydro-1H-indol-3-ylmethyl)-amide (40 mg), DIPEA (0.04 ml) and [2-fluoro-4-(2-oxo-2H-pyridin-1-yl)-phenyl]carbamic acid-4-nitro-phenyl ester (55 mg; prepared from 1-(4-amino-3-fluoro-phenyl)-1H-pyridin-2-one by reaction with 4-nitrophenyl-chlorformiat and pyridine in dichloromethane) in 5 ml DMF was heated 1.5 hrs at 80° C. The reaction mixture was diluted with AcOEt and washed with 1M NaOH and brine. The organic layers were dried over magnesium sulfate, evaporated and chromatographed (silica gel, AcOEt) to yield (3S)3-{[(5-chloro-thiophene-2-carbonyl)-amino]-methyl}-2,3-dihydro-indole-1-carboxylic acid [2-fluoro-4-(2-oxo-2H-pyridin-1-yl)-phenyl]-amide as a brown solid (72 mg). MS: 523.0 (M+H⁺)⁺.

### H (3R)-3-{[(5-Chloro-thiophene-2-carbonyl)-amino]-methyl}-2,3-dihydro-indole-1-carboxylic acid [2-fluoro-4-(2-oxo-2H-pyridin-1-yl)-phenyl]-amide

The title compound was obtained from (5S)-5-chloro-thiophene-2-carboxylic acid (2,3-dihydro-1H-indol-3-ylmethyl)-amide with the same procedure described in 38G) as a brown solid (73 mg). MS: 523.0 (M+H⁺)⁺.

### Example 39

### 3-{[(5-Chloro-thiophene-2-carbonyl)-amino]-methyl}-5,6-dimethoxy-2,3-dihydro-indole-1-carboxylic acid [2-fluoro-4-(2-oxo-2H-pyridin-1-yl)-phenyl]-amide

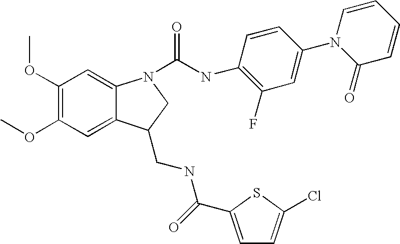

### A Benzyl-(5,6-dimethoxy-1H-indol-3-ylmethyl)-amine

A solution of the commercial 5,6-dimethoxy-1H-indole-3-carbaldehyde (1.5 g; CAS 142769-27-5) and benzylamine (1.2 ml) in 30 ml methanol was refluxed during 2 h, then cooled and natriumborhydride (415 mg) was added. After stirring 0.5 hrs under ice cooling, the reaction mixture was poured onto ice/water and the methanol evaporated. The brown residue was shaken with dichloromethane and NaHCO₃, the organic phase was washed with brine, dried over magnesium sulfate and concentrated followed by a precipitation (tBuOMe/heptane) of benzyl-(5,6-dimethoxy-1H-indol-3-ylmethyl)-amine as a white solid (2.02 g). MS: 297.1 (M+H⁺)⁺.

### B Benzyl-(5,6-dimethoxy-2,3-dihydro-1H-indol-3-ylmethyl)-amine

To a solution of benzyl-(5,6-dimethoxy-1H-indol-3-ylmethyl)-amine (1 g) in 35 ml THF at rt, was added natrium borohydride (638 mg). The mixture was heated to reflux, treated with bortrifluorid-ethyletherate (0.425 ml) and stirred 0.75 hrs under refluxing. After a complete evaporation, the crude product was treated with 1.25 M HCl/ethanol (60 ml) and stirred at reflux for one hour. The reaction mixture was concentrated, diluted with water and basified (with NaOH). The aqueous phase was extracted twice with dichloromethane. The organic layers were dried magnesium sulfate, evaporated and chromatographed (silica gel, dichloromethane/methanol, 9/1) to yield a brown oil (867 mg) of benzyl-(5,6-dimethoxy-2,3-dihydro-1H-indol-3-ylmethyl)-amine. MS: 299.2 (M+H⁺)⁺.

### C C-(5,6-Dimethoxy-2,3-dihydro-1H-indol-3-yl)-methylamine

Hydrogenation of benzyl-(5,6-dimethoxy-2,3-dihydro-1H-indol-3-ylmethyl)-amine (500 mg) in 10 ml methanol with Pd/C 10% (50 mg) at rt and filtration (decalite) yielded a brown oil (346 mg) of C-(5,6-dimethoxy-2,3-dihydro-1H-indol-3-yl)-methylamine. MS: 209.0 (M+H⁺)⁺.

### D 5-Chloro-thiophene-2-carboxylic acid (5,6-dimethoxy-2,3-dihydro-1H-indol-3-ylmethyl)-amide

To a cooled solution of C-(5,6-dimethoxy-2,3-dihydro-1H-indol-3-yl)-methylamine (50 mg) and DIPEA (0.123 ml) in 3 ml acetonitrile, were added successively EDC (69 mg), HOBT (49 mg) and 5-chloro-2-thiophenecarboxylic acid (39 mg). The reaction mixture was stirred 18 hrs at rt. Extraction (1M NaOH, brine/CH₂Cl₂) and chromatography (silica gel; AcOEt/methanol, 19/1) delivered 5-chloro-thiophene-2-carboxylic acid (5,6-dimethoxy-2,3-dihydro-1H-indol-3-ylmethyl)-amide as a yellow solid (34 mg). MS: 353.3 (M+H⁺)⁺.

### E 3-{[(5-Chloro-thiophene-2-carbonyl)-amino]-methyl}-5,6-dimethoxy-2,3-dihydro-indole-1-carboxylic acid [2-fluoro-4-(2-oxo-2H-pyridin-1-yl)-phenyl]-amide

In analogy to example 38G, 3-{[(5-chloro-thiophene-2-carbonyl)-amino]-methyl}-5,6-dimethoxy-2,3-dihydro-indole-1-carboxylic acid [2-fluoro-4-(2-oxo-2H-pyridin-1-yl)-phenyl]-amide (18 mg) as a brown solid was obtained from 5-chloro-thiophene-2-carboxylic acid (5,6-dimethoxy-2,3-dihydro-1H-indol-3-ylmethyl)-amide (30 mg). MS: 583.4 (M+H⁺)⁺.

### Example 40

### 3-{[(5-Chloro-thiophene-2-carbonyl)-amino]-methyl}-1-[2-fluoro-4-(2-oxo-2H-pyridin-1-yl)-phenylcarbamoyl]-2,3-dihydro-1H-indole-6-carboxylic acid methyl ester

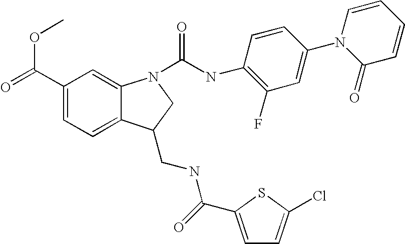

Starting from the commercial methyl 3-formylindole-6-carboxylate (CAS 133831-28-4) and using the same procedure described in example 39, 3-{[(5-chloro-thiophene-2-carbonyl)-amino]-methyl}-1-[2-fluoro-4-(2-oxo-2H-pyridin-1-yl)-phenylcarbamoyl]-2,3-dihydro-1H-indole-6-carboxylic acid methyl ester was obtained as a white solid (56 mg). MS: 581.2 (M+H⁺)⁺.

### Example 41

### 3-{[(5-Chloro-thiophene-2-carbonyl)-amino]-methyl}-2,3-dihydro-indole-1,6-dicarboxylic acid 6-dimethylamide 1-{[2-fluoro-4-(2-oxo-2H-pyridin-1-yl)-phenyl]-amide}

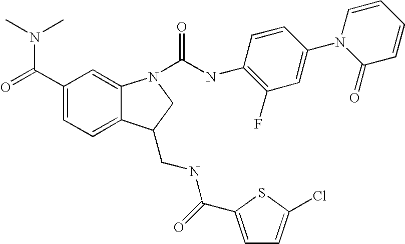

### A 3-{[(5-Chloro-thiophene-2-carbonyl)-amino]-methyl}-2,3-dihydro-1H-indole-6-carboxylic acid

The saponification of 3-{[(5-chloro-thiophene-2-carbonyl)-amino]-methyl}-2,3-dihydro-1H-indole-6-carboxylic acid methyl ester (68 mg; synthesized with a similar procedure described in example 39A-39D) was made with lithium hydroxide (9 mg) in 2 ml THF, 1 ml MeOH and 0.5 ml water. The reaction mixture was stirred 72 hrs at rt, diluted with dichloromethane, treated with magnesium sulfate, filtrated and evaporated to yield a light yellow solid (77 mg). MS: 335.3 (M−H)⁻.

### B 3-{[(5-Chloro-thiophene-2-carbonyl)-amino]-methyl}-2,3-dihydro-1H-indole-6-carboxylic acid dimethylamide

To a suspension of 3-{[(5-chloro-thiophene-2-carbonyl)-amino]-methyl}-2,3-dihydro-1H-indole-6-carboxylic acid (77 mg) in 3 ml acetonitrile, were added successively dimethylamine, HCl (74 mg), EDCI (65 mg), HOBT (46 mg) and DIPEA (0.23 ml). The reaction mixture was stirred 18 hrs at rt, diluted with dichloromethane, washed with 1M NaOH and brine. The organic phases were dried over magnesium sulfate and evaporated to give a yellow gum (49 mg). MS: 364.1 (M+H⁺)⁺.

### C 3-{[(5-Chloro-thiophene-2-carbonyl)-amino]-methyl}-2,3-dihydro-indole-1,6-dicarboxylic acid 6-dimethylamide 1-{[2-fluoro-4-(2-oxo-2H-pyridin-1-yl)-phenyl]-amide}

Starting from 3-{[(5-chloro-thiophene-2-carbonyl)-amino]-methyl}-2,3-dihydro-1H-indole-6-carboxylic acid dimethylamide (49 mg) and using the procedure described in example 38, the title compound was delivered as a white solid (23 mg). MS: 594.2 (M+H⁺)⁺.

### Example 42

### 5-Chloro-thiophene-2-carboxylic acid (1-{[2-fluoro-4-(2-oxo-2H-pyridin-1-yl)-phenylcarbamoyl]-methyl}-2,3-dihydro-1H-indol-3-yl)-amide

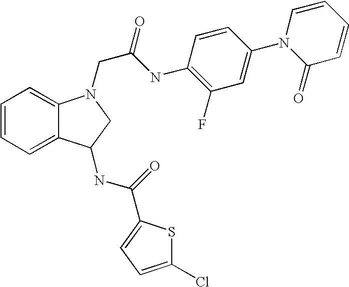

### A 2,3-Dihydro-indole-1,3-dicarboxylic acid 1-tert-butyl ester

2,3-Dihydro-indole-1,3-dicarboxylic acid 1-tert-butyl ester 3-methyl ester (4.2 g; CAS 528862-00-2; Tetrahedron 59(6), 747, 2003) in 30 ml THF, 15 ml methanol and 5 ml water was treated with lithium hydroxide (2 g) during 18 hrs at rt. The reaction mixture was washed with 1M HCl and brine. The organic layers were dried over magnesium sulfate, evaporated and chromatographed (silica gel, AcOEt) to deliver 2,3-dihydro-indole-1,3-dicarboxylic acid 1-tert-butyl ester as a white solid (3.6 g). MS: 264.3 (M+H⁺)⁺.

### B 3-Amino-2,3-dihydro-indole-1-carboxylic acid tert-butyl ester

To a solution of 2,3-dihydro-indole-1,3-dicarboxylic acid 1-tert-butyl ester (540 mg) in 5 ml dioxane heated at 80° C., were added via a syringe DIPEA (0.42 ml) and DPPA (0.52 ml). After heating at this temperature for 15 nm, the reaction mixture was poured onto 30 ml 1M KOH/crashed ice. Extraction (AcOEt) and chromatography (silica gel, AcOEt) delivered the title compound as a yellow solid (200 mg). MS: 235.2 (M+H⁺)⁺.

### C 3-[(5-Chloro-thiophene-2-carbonyl)-amino]-2,3-dihydro-indole-1-carboxylic acid tert-butyl ester

A solution of 3-amino-2,3-dihydro-indole-1-carboxylic acid tert-butyl ester (200 mg), 5-chloro-2-thiophencarboxylic acid (162 mg), DIPEA (0.40 ml) and BOP-Cl (254 mg) in 2 ml acetonitrile and 0.2 ml DMF was stirred 1 hr at rt. The reaction mixture was diluted with AcOEt, washed with 1M HCl, 1M NaOH and brine. The organic layers were dried over magnesium sulfate, evaporated, and chromatographed (silica gel, AcOEt/heptane, 1/1) to yield 3-[(5-chloro-thiophene-2-carbonyl)-amino]-2,3-dihydro-indole-1-carboxylic acid tert-butyl ester as a white solid (264 mg). MS: 396.1 (M+NH₄⁺)⁺.

### D 5-Chloro-thiophene-2-carboxylic acid (2,3-dihydro-1H-indol-3-yl)-amide

A solution of 3-[(5-chloro-thiophene-2-carbonyl)-amino]-2,3-dihydro-indole-1-carboxylic acid tert-butyl ester in 10 ml TFA was stirred 2 hrs at rt and evaporated. Extraction (1M NaOH/AcOEt) and chromatography (silica gel; AcOEt) delivered the title compound as a white solid (523 mg). MS: 279.1 (M+H⁺)⁺.

### E 5-Chloro-thiophene-2-carboxylic acid (1-{[2-fluoro-4-(2-oxo-2H-pyridin-1-yl)-phenylcarbamoyl]-methyl}-2,3-dihydro-1H-indol-3-yl)-amide

To a cooled solution of 5-chloro-thiophene-2-carboxylic acid (2,3-dihydro-1H-indol-3-yl)-amide (90 mg) in 2 ml THF, were added NaH (50 mg) and 2-bromo-N-[2-fluoro-4-(2-oxo-2H-pyridin-1-yl)-phenyl]-acetamide (100 mg; prepared from 1-(4-amino-3-fluoro-phenyl)-1H-pyridin-2-one by reaction with bromo-acetylbromid and DIPEA in dichloromethane). The reaction mixture was stirred 1 hr at 0° C. and 18 hrs at rt, diluted with AcOEt and extracted with water and brine. The organic layers were dried over magnesium sulfate, evaporated and chromatographed (silica gel; AcOEt) to yield 5-chloro-thiophene-2-carboxylic acid (1-{[2-fluoro-4-(2-oxo-2H-pyridin-1-yl)-phenylcarbamoyl]-methyl}-2,3-dihydro-1H-indol-3-yl)-amide as a yellow solid (34 mg). MS: 523.0 (M+H⁺)⁺.

### Example 43

### 3-{[(5-Chloro-thiophene-2-carbonyl)-amino]-methyl}-4-methyl-2,3-dihydro-indole-1-carboxylic acid [2-fluoro-4-(2-oxo-2H-pyridin-1-yl)-phenyl]-amide

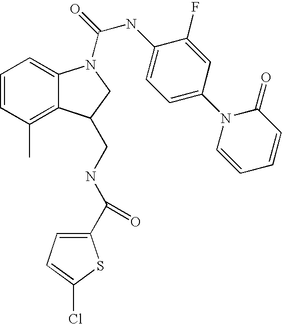

### A (2-Bromo-3-methyl-phenyl)-carbamic acid tert-butyl ester

2-Bromo-3-methyl-phenylamine (1.06 g; CAS 54879-20-8), Boc₂O (3.73 g) and DMAP (69 mg) in 50 ml THF were refluxed 2 hrs. The reaction mixture was concentrated, followed by an acidic extraction. The combined organic phases were dried over magnesium sulfate and evaporated. The crude product was taken in 50 ml methanol and K₂CO₃ (2.36 g) was added. The suspension was heated to reflux 2 h, and at rt for 18 h, evaporated and extracted with 0.5M HCl/AcOEt. The organic layers were dried over magnesium sulfate and chromatographed (silica gel; AcOEt/heptane, 1/9) to deliver a yellow oil (1.38 g). MS: 285/287 (M+H⁺)⁺.

### B (2-Bromo-3-methyl-phenyl)-(3-chloro-allyl)-carbamic acid tert-butyl ester

(2-Bromo-3-methyl-phenyl)-carbamic acid tert-butyl ester (1.2 g) was dissolved in 12 ml THF and treated successively with tetrabutylammonium bromide (67 mg), 1,3-dichloropropene (1.95 ml) and stirred under argon at 0° C. Then, natrium hydride (275 mg) was carefully added. After 2 hrs at 0° C. and 2 hrs at rt., the reaction mixture was poured onto 10% NH₄Cl, twofold extracted with AcOEt, dried over magnesium sulfate, and purified by chromatography (silica gel; AcOEt/heptane, 1/9). (2-Bromo-3-methyl-phenyl)-(3-chloro-allyl)-carbamic acid tert-butyl ester was obtained as a yellow oil (1.55 g). MS: 270/272 (M−Cl, isobutylene)⁺; 305/307 (M−isobutylene)⁺; pic absent 360 (M)⁺.

### C 3-Chloromethyl-4-methyl-2,3-dihydro-indole-1-carboxylic acid tert-butyl ester

During one hour, argon was bubbed through a solution of (2-bromo-3-methyl-phenyl)-(3-chloro-allyl)-carbamic acid tert-butyl ester (980 mg), AIBN (22 mg) and tri-n-butyltin hydride (0.79 ml) in 120 ml benzene. The reaction mixture was refluxed 3 hrs, evaporated to dryness and chromatographed (silica gel; AcOEt/heptane, 1/9) to yield 3-chloromethyl-4-methyl-2,3-dihydro-indole-1-carboxylic acid tert-butyl ester as a colorless oil (327 mg). MS: 281.3 (M)⁺.

### D 3-Azidomethyl-1,4-dimethyl-2,3-dihydro-indole-1-carboxylic acid tert-butyl ester

3-Chloromethyl-4-methyl-2,3-dihydro-indole-1-carboxylic acid tert-butyl ester (307 mg) was dissolved in 3 ml DMF and treated with natrium azid (106 mg) 18 hrs at 60° C. The reaction mixture was diluted with AcOEt, washed with 1M NaOH and brine. The organic layers were dried over magnesium sulfate, evaporated and purified by chromatography (silica gel; AcOEt/heptane, 1/9) to deliver a white solid (200 mg). MS: 288.2 (M)⁺.

### E 3-Aminomethyl-1,4-dimethyl-2,3-dihydro-indole-1-carboxylic acid tert-butyl ester

A solution of 3-azidomethyl-1,4-dimethyl-2,3-dihydro-indole-1-carboxylic acid tert-butyl ester (190 mg) and triphenylphosphine (172 mg) in 5 ml THF was stirred 2 hrs at 60° C. A solution of ammonium hydroxide (2 ml) was added and the reaction mixture kept 18 hrs at rt. An extraction with 1M NaOH and brine followed by a chromatography (silica gel, AcOEt/MeOH 9/1 to 3/1) gave the title compound as a colorless oil (154 mg). MS: 262.9 (M+H)⁺.

### F 3-{[(5-Chloro-thiophene-2-carbonyl)-amino]-methyl}-1,4-dimethyl-2,3-dihydro-indole-1-carboxylic acid tert-butyl ester

Starting from 3-aminomethyl-1,4-dimethyl-2,3-dihydro-indole-1-carboxylic acid tert-butyl ester (193 mg) and using the procedure described in example 38E, the title compound was obtained a white residue (205 mg). MS: 407.1 (M+H)⁺.

### G 5-Chloro-thiophene-2-carboxylic acid (1,4-dimethyl-2,3-dihydro-1H-indol-3-ylmethyl)-amide

3-{[(5-Chloro-thiophene-2-carbonyl)-amino]-methyl}-1,4-dimethyl-2,3-dihydro-indole-1-carboxylic acid tert-butyl ester (193 mg) was treated with 5 ml dichloromethane and 1.1 ml TFA 2 hrs at rt. The reaction mixture was poured onto 1M NaOH/ice and threefold extracted with dichloromethane. The organic layers were dried over magnesium sulfate and evaporated to deliver a white foam (128 mg). MS: 307.0 (M+H)⁺.

### H 3-{[(5-Chloro-thiophene-2-carbonyl)-amino]-methyl}-4-methyl-2,3-dihydro-indole-1-carboxylic acid [2-fluoro-4-(2-oxo-2H-pyridin-1-yl)-phenyl]-amide

Starting from 5-chloro-thiophene-2-carboxylic acid (1,4-dimethyl-2,3-dihydro-1H-indol-3-ylmethyl)-amide (60 mg) and using the procedure described in example 38G, the title compound was obtained as a white solid (98 mg). MS: 537.2 (M+H)⁺.

### Example 44

### 4-Chloro-3-{[(5-chloro-thiophene-2-carbonyl)-amino]-methyl}-2,3-dihydro-indole-1-carboxylic acid [2-fluoro-4-(2-oxo-2H-pyridin-1-yl)-phenyl]-amide

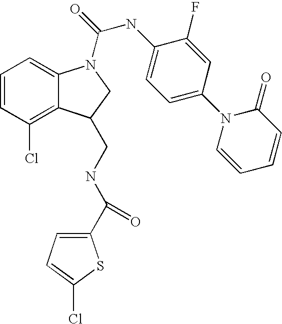

### A 2-Bromo-3-chloro-phenylamine

Starting from 2-bromo-3-chloro-benzoic acid (500 mg; CAS 56961-26-3) dissolved in 5 ml toluene, was treated with triethylamine (0.3 ml), diphenylphosphorylazide (0.69 ml) and t-butanol (3.6 ml) and heated 2 hrs at 80° C. Additional tert-butanol (3.6 ml) was added and the solution stirred 18 hrs at 100° C. The reaction mixture was evaporated to dryness and chromatographed (silica gel; AcOEt/heptane, 1/19) to yield 2-bromo-3-chloro-phenylamine as a white solid (410 mg). MS: 305/307 (M+H⁺)⁺.

### B (4-Chloro-3-{[(5-chloro-thiophene-2-carbonyl)-amino]-methyl}-2,3-dihydro-indole-1-carboxylic acid [2-fluoro-4-(2-oxo-2H-pyridin-1-yl)-phenyl]-amide

Starting from the above compound and using the same sequence of steps described in example 43A-43H, 4-chloro-3-{[(5-chloro-thiophene-2-carbonyl)-amino]-methyl}-2,3-dihydro-indole-1-carboxylic acid [2-fluoro-4-(2-oxo-2H-pyridin-1-yl)-phenyl]-amide was obtained as a white solid (144 mg). MS: 557.0 (M+H⁺)⁺.

### Example 45

### 3-{[(5-Chloro-thiophene-2-carbonyl)-amino]-methyl}-2,3-dihydro-indole-1,5-dicarboxylic acid 5-dimethylamide 1-{[2-fluoro-4-(2-oxo-2H-pyridin-1-yl)-phenyl]amide}

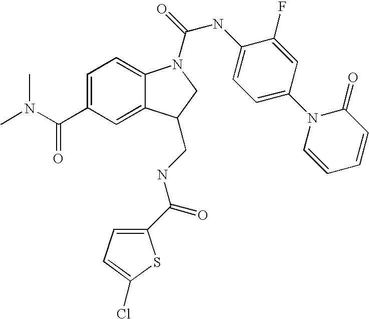

Starting from 4-amino-3-iodo-benzoic acid methyl ester (25 g) and using the sequence of steps described in example 43A-43F, the intermediate 3-{[(5-chloro-thiophene-2-carbonyl)-amino]-methyl}-2,3-dihydro-indole-1,5-dicarboxylic acid 1-tert-butyl ester 5-methyl ester was obtained as a colorless oil (270 mg). This product was treated successively with similar procedures described in examples 41A-41B and then 38F-38G to yield the title compound 3-{[(5-chloro-thiophene-2-carbonyl)-amino]-methyl}-2,3-dihydro-indole-1,5-dicarboxylic acid 5-dimethylamide 1-{[2-fluoro-4-(2-oxo-2H-pyridin-1-yl)-phenyl]amide}as a white solid (37 mg). MS: 594.3 (M+H⁺)⁺.

### Example A

Film coated tablets containing the following ingredients can be manufactured in a conventional manner:

| Ingredients | Per tablet | |
|---|---|---|
| Kernel: | | |
| Compound of formula (I) | 10.0 mg | 200.0 mg |
| Microcrystalline cellulose | 23.5 mg | 43.5 mg |
| Lactose hydrous | 60.0 mg | 70.0 mg |
| Povidone K30 | 12.5 mg | 15.0 mg |
| Sodium starch glycolate | 12.5 mg | 17.0 mg |
| Magnesium stearate | 1.5 mg | 4.5 mg |
| (Kernel Weight) | 120.0 mg | 350.0 mg |
| Film Coat: | | |
| Hydroxypropyl methyl cellulose | 3.5 mg | 7.0 mg |
| Polyethylene glycol 6000 | 0.8 mg | 1.6 mg |
| Talc | 1.3 mg | 2.6 mg |
| Iron oxyde (yellow) | 0.8 mg | 1.6 mg |
| Titan dioxide | 0.8 mg | 1.6 mg |

The active ingredient is sieved and mixed with microcristalline cellulose and the mixture is granulated with a solution of polyvinylpyrrolidon in water. The granulate is mixed with sodium starch glycolate and magesiumstearate and compressed to yield kernels of 120 or 350 mg respectively. The kernels are lacquered with an aqueous solution/suspension of the above mentioned film coat.

### Example B

Capsules containing the following ingredients can be manufactured in a conventional manner:

| Ingredients | Per capsule |
|---|---|
| Compound of formula (I) | 25.0 mg |
| Lactose | 150.0 mg |
| Maize starch | 20.0 mg |
| Talc | 5.0 mg |

The components are sieved and mixed and filled into capsules of size 2.

### Example C

Injection solutions can have the following composition:

| | |
|---|---|
| Compound of formula (I) | 3.0 mg |
| Polyethylene Glycol 400 | 150.0 mg |
| Acetic Acid | q.s. ad pH 5.0 |
| Water for injection solutions | ad 1.0 ml |

The active ingredient is dissolved in a mixture of Polyethylene Glycol 400 and water for injection (part). The pH is adjusted to 5.0 by Acetic Acid. The volume is adjusted to 1.0 ml by addition of the residual amount of water. The solution is filtered, filled into vials using an appropriate overage and sterilized.

### Example D

Soft gelatin capsules containing the following ingredients can be manufactured in a conventional manner:

| | |
|---|---|
| Capsule contents | |
| Compound of formula (I) | 5.0 mg |
| Yellow wax | 8.0 mg |
| Hydrogenated Soya bean oil | 8.0 mg |
| Partially hydrogenated plant oils | 34.0 mg |
| Soya bean oil | 110.0 mg |
| Weight of capsule contents | 165.0 mg |
| Gelatin capsule | |
| Gelatin | 75.0 mg |
| Glycerol 85% | 32.0 mg |
| Karion 83 | 8.0 mg |
| | (dry matter) |
| Titan dioxide | 0.4 mg |
| Iron oxide yellow | 1.1 mg |

The active ingredient is dissolved in a warm melting of the other ingredients and the mixture is filled into soft gelatin capsules of appropriate size. The filled soft gelatin capsules are treated according to the usual procedures.

### Example E

Sachets containing the following ingredients can be manufactured in a conventional manner:

| | |
|---|---|
| Compound of formula (I) | 50.0 mg |
| Lactose, fine powder | 1015.0 mg |
| Microcristalline cellulose (AVICEL PH 102) | 1400.0 mg |
| Sodium carboxymethyl cellulose | 14.0 mg |
| Polyvinylpyrrolidon K 30 | 10.0 mg |
| Magnesiumstearate | 10.0 mg |
| Flavoring additives | 1.0 mg |

The active ingredient is mixed with lactose, microcristalline cellulose and sodium carboxymethyl cellulose and granulated with a mixture of polyvinylpyrrolidon in water. The granulate is mixed with magnesiumstearate and the flavouring additives and filled into sachets.

What is claimed is:

1. A compound of formula (I) 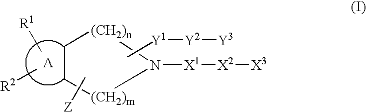
wherein
A is a carbocyclic ring which is a monocyclic or bicyclic aromatic ring of 5 to 12 ring atoms, or a monocyclic or bicyclic non-aromatic ring of 5 to 12 ring atoms, wherein one or two carbon atoms of said carbocyclic ring are optionally replaced with a carbonyl group;
R¹ and R² are independently hydrogen, C₁₋₆alkyl, C₁₋₆alkoxy, fluoro C₁₋₆alkoxy, hydroxy C₁₋₆ alkoxy, C₁₋₆alkoxy C₁₋₆ alkoxy, C₁₋₆ alkoxycarbonyl, mono- or di-C₁₋₆alkyl substituted amino C₁₋₆alkoxy, halogen, cyano, nitro, —N(R′)—CO—(C₁₋₆alkyl optionally substituted by one or more fluorine atoms), wherein R′ is hydrogen, C₁₋₆alkyl or fluoro C₁₋₆alkyl, —N(R′)—CO—O—(C₁₋₆alkyl optionally substituted by one or more fluorine atoms), wherein R′ is hydrogen, C₁₋₆alkyl or fluoro C₁₋₆alkyl, —N(R′)—CO—N(R″) (R′″), wherein R′, R″ and R′″ are independently hydrogen, C₁₋₆alkyl or fluoro C₁₋₆ alkyl or —N(R′)—SO₂—(C₁₋₆alkyl optionally substituted by one or more fluorine atoms), wherein R′ is hydrogen, C₁₋₆alkyl or fluoro C₁₋₆ alkyl or
R¹ and R² are independently —SO₂—N(R′)(R″), —C(O)—N(R′)(R″) or —N(R′)(R″), wherein R′ and R″ are independently hydrogen, C₁₋₆alkyl or fluoro C₁₋₆alkyl or R′ and R″, together with the nitrogen atom to which they are attached, form heterocycyl;
X¹ is —C(O)—(C₀₋₆alkylene)-NR³—(C₀₋₆alkylene)-, —(C₀₋₆alkylene)-C(O)—NR³—(C₀₋₆alkylene)-, —(C₁₋₆alkylene)-NR³—C(O)—(C₀₋₆alkylene)-, —C(O)—(C₀₋₆alkylene)-, C₀₋₆alkylene, —SO₂—(C₀₋₆alkylene)-, —(C₀₋₆alkylene)-SO₂—NR³—(C₀₋₆alkylene)- or 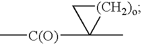
X² is arylene, heteroarylene or heterocyclylene, said arylene, heteroarylene and heterocyclylen being optionally substituted by one or more substituents independently selected from the group consisting of C₁₋₆alkyl, C₁₋₆ alkoxy, halogen, cyano, nitro, amino, —N(R′)—CO—(C₁₋₆alkyl optionally substituted by one or more fluorine atoms), wherein R′ is hydrogen, C₁₋₆alkyl or fluoro C₁₋₆ alkyl, —N(R′)—CO—O—(C₁₋₆alkyl optionally substituted by one or more fluorine atoms), wherein R′ is hydrogen, C₁₋₆alkyl or fluoro C₁₋₆ alkyl, —N(R′)—CO—N(R″)(R′″), wherein R′, R″ and R′″ are independently hydrogen, C₁₋₆alkyl or fluoro C₁₋₆ alkyl, —C(O)—N(R′)(R″), wherein R′ and R″ are independently hydrogen, C₁₋₆alkyl or fluoro C₁₋₆ alkyl, or R′ and R″, together with the nitrogen atom to which they are attached, form heterocycyl, —NR′R″, wherein R′ and R″ are independently hydrogen, C₁₋₆alkyl or fluoro C₁₋₆alkyl, or R′ and R″, together with the nitrogen atom to which they are attached, form heterocycyl, 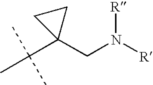
wherein R′ and R″ are independently C₁₋₆alkyl or fluoro C₁₋₆alkyl, or R′ and R″, together with the nitrogen atom to which they are attached, form heterocyclyl, 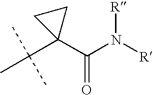
wherein R′ and R″ are independently C₁₋₆alkyl or fluoro C₁₋₆alkyl, or R′ and R″, together with the nitrogen atom to which they are attached, form heterocyclyl, 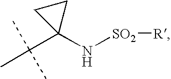
wherein R′ is fluoro C₁₋₆alkyl and 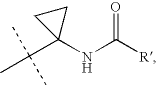
wherein R′ is fluoro C₁₋₆alkyl,
and one or two carbon atoms of said arylene, heteroarylene or heterocyclylene being optionally replaced with a carbonyl group;
X³ is hydrogen, aryl, heteroaryl or heterocyclyl, said aryl, heteroaryl and heterocyclyl being optionally substituted by one or more substituents independently selected from the group consisting of C₁₋₆alkyl, C₁₋₆alkoxy, halogen, cyano, nitro, amino, mono-C₁₋₆alkyl substituted amino, di-C₁₋₆alkyl substituted amino, mono-C₁₋₆alkyl substituted amino-C₁₋₆alkyl, di-C₁₋₆alkyl substituted amino-C₁₋₆alkyl, —SO₂—C₁₋₆alkyl, —SO₂—NH₂, —SO₂—NH—C₁₋₆alkyl and —SO₂—N(C₁₋₆alkyl)₂,
and one or two carbon atoms of said aryl, heteroaryl and heterocyclyl being optionally replaced with a carbonyl group;
R³ is hydrogen or C₁₋₆alkyl;
Y¹ is —(C₀₋₆alkylene)-C(O)—NR³—(C₀₋₆alkylene)-, —(C₀₋₆alkylene)-NR³—C(O)—(C₀₋₆alkylene)-, —C(O)—(C₀₋₆alkylene)- or C₀₋₆alkylene;
Y² is arylene, heteroarylene or heterocyclylene, said arylene, heteroarylene and heterocyclylene being optionally substituted by one or more substituents independently selected from the group consisting of C₁₋₆alkyl, C₁₋₆ alkoxy, halogen, cyano, nitro, amino, —N(R′)—CO—(C₁₋₆alkyl optionally substituted by one or more fluorine atoms), wherein R′ is hydrogen, C₁₋₆alkyl or fluoro C₁₋₆ alkyl, —N(R′)—CO—O—(C₁₋₆alkyl optionally substituted by one or more fluorine atoms), wherein R′ is hydrogen, C₁₋₆alkyl or fluoro C₁₋₆ alkyl, —N(R′)—CO—N(R″) (R′″), wherein R′, R″ and R′″ are independently hydrogen, C₁₋₆alkyl or fluoro C_{1-6alkyl}, —C(O)—N(R′)(R″), wherein R′ and R″ are independently hydrogen, C₁₋₆alkyl or halo C₁₋₆alkyl, or R′ and R″, together with the nitrogen atom to which they are attached, form heterocycyl, —NR′R″, wherein R′ and R″ are independently hydrogen, C₁₋₆alkyl or halo C₁₋₆alkyl, or R′ and R″, together with the nitrogen atom to which they are attached, form heterocycyl, 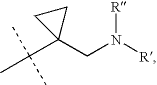
wherein R′ and R″ are independently C₁₋₆alkyl or fluoro C₁₋₆alkyl, or R′ and R″, together with the nitrogen atom to which they are attached, form heterocyclyl, 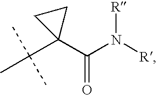
wherein R′ and R″ are independently C₁₋₆alkyl or fluoro C₁₋₆alkyl, or R′ and R″, together with the nitrogen atom to which they are attached, form heterocyclyl, 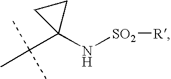
wherein R′ is fluoro C₁₋₆alkyl and 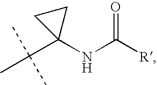
wherein R′ is C₁₋₆alkyl,
and one or two carbon atoms of said arylene, heteroarylene or heterocyclylene being optionally replaced with a carbonyl group;
Y³ is hydrogen, aryl, heteroaryl or heterocyclyl, said aryl, heteroaryl and heterocyclyl being optionally substituted by one or more substituents independently selected from the group consisting of C₁₋₆alkyl, C₁₋₆ alkoxy, halogen, cyano, nitro, amino, mono-C₁₋₆alkyl substituted amino, di-C₁₋₆alkyl substituted amino, mono-C₁₋₆alkyl substituted amino-C₁₋₆alkyl, di-C₁₋₆alkyl substituted amino-C₁₋₆alkyl, —SO₂—C₁₋₆alkyl, —SO₂—NH₂, —SO₂—NH—C₁₋₆alkyl and —SO₂—N(C₁₋₆alkyl)₂, and one or two carbon atoms of said aryl, heteroaryl and heterocyclyl being optionally replaced with a carbonyl group;
Z is attached to the same carbon atom as —¹—Y²—Y³, and hydrogen or C₁₋₆alkyl;
n is 0, 1 or 2;
m is 0, 1 or 2;
m+n is 2 or 3;
o is an integer from 1 to 5;
and prodrugs and pharmaceutically acceptable salts thereof.

2. A compound according to claim 1 with the formula

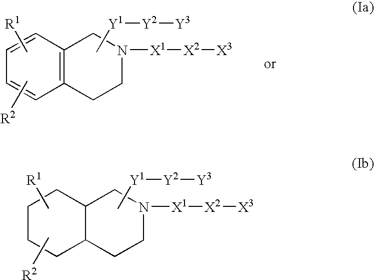

3. A compound according to claim 2, wherein X¹ is —(C₀₋₆alkylene)-C(O)—NH—.

4. A compound according to claim 2, wherein X¹ is —C(O)—NH—.

5. A compound according to claim 2 wherein X² is arylene or heteroarylene, said arylene and heteroarylene being optionally substituted by one or more substituents independently selected from the group consisting of C₁₋₆ alkoxy and halogen, and X³ is hydrogen.

6. A compound according to claim 2 wherein —X²—X³ forms phenyl or pyridyl, said phenyl and pyridyl being optionally substituted by one or more of the same or different halogen atoms.

7. A compound according to claim 6, wherein —X²—X³ forms 4-chlorophenyl.

8. A compound according to claim 2 wherein Y¹ is —(C₀₋₆alkylene)-C(O)—NH—.

9. A compound according to claim 8, wherein Y¹ is —C(O)—NH—.

10. A compound according to claim 2, wherein Y² is 1,4-phenylene optionally substituted by one or more same or different halogen atoms.

11. A compound according to claim 10, wherein Y² is 2-fluoro-1,4 phenylene.

12. A compound according to claim 2, wherein Y³ is heteroaryl or heterocyclyl, said heteroaryl and heterocyclyl being optionally substituted by one or more substituents independently selected from the group consisting of C₁₋₆alkyl, C₁₋₆ alkoxy, halogen, cyano, nitro, amino, mono-C₁₋₆alkyl substituted amino, di-C₁₋₆alkyl substituted amino, mono-C₁₋₆alkyl substituted amino-C₁₋₆alkyl, di-C₁₋₆alkyl substituted amino-C₁₋₆alkyl, —SO₂—C₁₋₆alkyl, —SO₂—NH₂, —SO₂—NH—C₁₋₆alkyl and —SO₂—N(C₁₋₆alkyl)₂, wherein one or two carbon atoms of said heteroaryl and heterocyclyl are optionally replaced with a carbonyl group.

13. A compound according to claim 12, wherein Y³ is 2-oxo-2H-pyridyn-1-yl.

14. A compound according to claim 2, wherein —Y¹—Y²—Y³ is bonded to 3 position of the isoquinoline ring.

15. A compound according to claim 2, wherein R¹ and R² are hydrogen.

16. A compound according to claim 1 with the formula

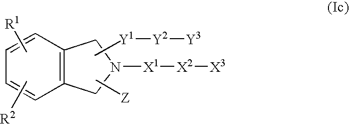

17. A compound according to claim 16, wherein X¹ is —(C₀₋₆alkylene)-C(O)—NH—.

18. A compound according to claim 17, wherein X¹ is —C(O)—NH—.

19. A compound according to claim 16, wherein X² is arylene or heteroarylene, said arylene and heteroarylene being optionally substituted by one or more substituents independently selected from the group consisting of C₁₋₆ alkoxy and halogen, and X³ is hydrogen.

20. A compound according to claim 16, wherein —X²—X³ forms phenyl or pyridyl, said phenyl and pyridyl being optionally substituted by one or more same or different halogen atoms.

21. A compound according to claim 20, wherein —X²—X³ forms 4-chlorophenyl or 5-chloro-2-pyridyl.

22. A compound according to claim 16, wherein Y¹ is —(C₀₋₆alkylene)-C(O)—NH—.

23. A compound according to claim 22, wherein Y¹ is —C(O)—NH—.

24. A compound according to claim 16, wherein Y² is 1,4-phenylene optionally substituted by one or more same or different halogen atoms.

25. A compound according to claim 24, wherein Y² is 2-fluoro-1,4 phenylene.

26. A compound according to claim 16, wherein Y³ is heteroaryl or heterocyclyl, said heteroaryl and heterocyclyl being optionally substituted by one or more substituents independently selected from the group consisting of C₁₋₆alkyl, C₁₋₆ alkoxy, halogen, cyano, nitro, amino, mono-C₁₋₆alkyl substituted amino, di-C₁₋₆alkyl substituted amino, mono-C₁₋₆alkyl substituted amino-C₁₋₆alkyl, di-C₁₋₆alkyl substituted amino-C₁₋₆alkyl, —SO₂—C₁₋₆alkyl, —SO₂—NH₂, —SO₂—NH—C₁₋₆alkyl and —SO₂—N(C₁₋₆alkyl)₂, wherein one or two carbon atoms of said heteroaryl and heterocyclyl are optionally replaced with a carbonyl group.

27. A compound according to claim 26, wherein Y³ is 2-oxo-2H-pyridyn-1-yl.

28. A compound according to claim 16, wherein —Y¹—Y²—Y³ is bonded to 1 position of the isoindole ring.

29. A compound according to claim 16, wherein R¹ and R² are hydrogen.

30. A compound according to claim 16, wherein Z is hydrogen or methyl.

31. A compound according to claim 1 with the formula

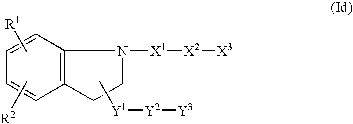

32. A compound according to claim 31, wherein X¹ is —(C₀₋₆alkylene)-C(O)—NH—.

33. A compound according to claim 32, wherein X¹ is —C(O)—NH—.

34. A compound according to claim 31, wherein X² is 1,4-phenylene optionally substituted by one or more of the same or different halogen atoms.

35. A compound according to claim 34, wherein X² is 2-fluoro-1,4 phenylene.

36. A compound according to claim 31, wherein X³ is heteroaryl which is optionally substituted by one or more substituents independently selected from the group consisting of C₁₋₆alkyl, C₁₋₆ alkoxy, halogen, cyano, nitro, amino, mono-C₁₋₆alkyl substituted amino, di-C₁₋₆alkyl substituted amino, mono-C₁₋₆alkyl substituted amino-C₁₋₆alkyl, di-C₁₋₆alkyl substituted amino-C₁₋₆alkyl, —SO₂—C₁₋₆alkyl, —SO₂—NH₂, —SO₂—NH—C₁₋₆alkyl and —SO₂—N(C₁₋₆alkyl)₂, wherein one or two carbon atoms of said heteroaryl are optionally replaced with a carbonyl group.

37. A compound according to claim 36, wherein X³ is 2-oxo-2H-pyridyn-1-yl.

38. A compound according to claim 31, wherein Y¹ is —(C₀₋₆alkylene)-NH—C(O)—.

39. A compound according to claim 38, wherein Y¹ is —CH₂—NH—C(O)—.

40. A compound according to claim 31, wherein Y² is heteroarylene which is optionally substituted by one or more same or different halogen atoms, and Y³ is hydrogen.

41. A compound according to claim 31, wherein —Y²—Y³ forms thienyl optionally substituted by one or more same or different halogen atoms.

42. A compound according to claim 41, wherein —Y²—Y³ forms 5-chloro-2-thienyl.

43. A compound according to claim 31, wherein —Y¹—Y²—Y³ is bonded to 3 position of the indole ring.

44. A compound according to claim 31, wherein one of R¹ and R² is hydrogen or C₁₋₆ alkoxy, and the other is selected from the group consisting of hydrogen, C₁₋₆alkyl, C₁₋₆ alkoxy, C₁₋₆ alkoxycarbonyl, halogen and —C(O)—N(R′)(R″), wherein R′ and R″ are independently hydrogen, C₁₋₆alkyl or fluoro C₁₋₆alkyl.

45. A compound according to claim 1 selected from the group consisting of:
1,3-Dihydro-isoindole-1,2-dicarboxylic acid 2-[(4-chloro-phenyl)-amide]1-{[2-fluoro-4-(2-oxo-2H-pyridin-1-yl)-phenyl]-amide},
1,3-Dihydro-isoindole-1,2-dicarboxylic acid 2-[(5-chloro-pyridin-2-yl)-amide]1-{[2-fluoro-4-(2-oxo-2H-pyridin-1-yl)-phenyl]-amide},
(S)-1,3-Dihydro-isoindole-1,2-dicarboxylic acid 2-[(4-chloro-phenyl)-amide]1-{[4-(2-oxo-2H-pyridin-1-yl)-phenyl]-amide},
(R)-1,3-Dihydro-isoindole-1,2-dicarboxylic acid 2-[(4-chloro-phenyl)-amide]1-{[4-(2-oxo-2H-pyridin-1-yl)-phenyl]-amide},
(R)-1,3-Dihydro-isoindole-1,2-dicarboxylic acid 2-[(5-chloro-pyridin-2-yl)-amide]1-{[4-(2-oxo-2H-pyridin-1-yl)-phenyl]-amide},
(S)-1,3-Dihydro-isoindole-1,2-dicarboxylic acid 2-[(5-chloro-pyridin-2-yl)-amide]1-{[4-(2-oxo-2H-pyridin-1-yl)-phenyl]-amide},
(S)-1,3-Dihydro-isoindole-1,2-dicarboxylic acid 2-[(4-chloro-phenyl)-amide]1-{[4-(2-oxo-2H-pyrazin-1-yl)-phenyl]-amide},
(R)-1,3-Dihydro-isoindole-1,2-dicarboxylic acid 2-[(4-chloro-phenyl)-amide]1-{[4-(2-oxo-2H-pyrazin-1-yl)-phenyl]-amide},
1,3-Dihydro-isoindole-1,2-dicarboxylic acid 2-[(4-chloro-phenyl)-amide]1-{[2-fluoro-4-(2-oxo-2H-pyrazin-1-yl)-phenyl]-amide},
1,3-Dihydro-isoindole-1,2-dicarboxylic acid 2-[(4-chloro-phenyl)-amide]1-{[2-fluoro-4-(3-oxo-morpholin-4-yl)-phenyl]-amide},
1-Methyl-1,3-dihydro-isoindole-1,2-dicarboxylic acid 2-[(4-chloro-phenyl)-amide]1-{[2-fluoro-4-(2-oxo-2H-pyridin-1-yl)-phenyl]-amide},
(R)-1-Methyl-1,3-dihydro-isoindole-1,2-dicarboxylic acid 2-[(4-chloro-phenyl)-amide]1-{[2-fluoro-4-(2-oxo-2H-pyridin-1-yl)-phenyl]-amide},
1,3-Dihydro-isoindole-1,2,6-tricarboxylic acid 2-[(4-chloro-phenyl)-amide]6-dimethylamide 1-{[2-fluoro-4-(2-oxo-2H-pyridin-1-yl)-phenyl]-amide},
(R)-3,4-Dihydro-1H-isoquinoline-2,3-dicarboxylic acid 2-[(4-chloro-phenyl)-amide]3-{[2-fluoro-4-(2-oxo-2H-pyridin-1-yl)-phenyl]-amide},
(S)-3,4-Dihydro-1H-isoquinoline-2,3-dicarboxylic acid 2-[(4-chloro-phenyl)-amide]3-{[2-fluoro-4-(2-oxo-2H-pyridin-1-yl)-phenyl]-amide},
(R)-3,4-Dihydro-1H-isoquinoline-2,3-dicarboxylic acid 2-[(4-chloro-phenyl)-amide]3-{[2-fluoro-4-(3-oxo-morpholin-4-yl)-phenyl]-amide},
(R)-3,4-Dihydro-1H-isoquinoline-2,3-dicarboxylic acid 2-[(5-chloro-pyridin-2-yl)-amide]3-{[2-fluoro-4-(2-oxo-2H-pyridin-1-yl)-phenyl]-amide},
(R)-3,4-Dihydro-1H-isoquinoline-2,3-dicarboxylic acid 3-{[2-fluoro-4-(2-oxo-2H-pyridin-1-yl)-phenyl]-amide}2-[(4-methoxy-phenyl)-amide],
(R)-3,4-Dihydro-1H-isoquinoline-2,3-dicarboxylic acid 3-[(4-chloro-phenyl)-amide]2-{[2-fluoro-4-(2-oxo-2H-pyridin-1-yl)-phenyl]-amide},
(R)-Octahydro-isoquinoline-2,3-dicarboxylic acid 2-[(4-chloro-phenyl)-amide]3-{[2-fluoro-4-(2-oxo-2H-pyridin-1-yl)-phenyl]-amide},
(R)-3-{[(5-Chloro-thiophene-2-carbonyl)-amino]-methyl}-2,3-dihydro-indole-1-carboxylic acid
[2-fluoro-4-(2-oxo-2H-pyridin-1-yl)-phenyl]-amide,
(S)-3-{[(5-Chloro-thiophene-2-carbonyl)-amino]-methyl}-2,3-dihydro-indole-1-carboxylic acid [2-fluoro-4-(2-oxo-2H-pyridin-1-yl)-phenyl]-amide,
3-{[(5-Chloro-thiophene-2-carbonyl)-amino]-methyl}-1-[2-fluoro-4-(2-oxo-2H-pyridin-1-yl)-phenylcarbamoyl]-2,3-dihydro-1H-indole-6-carboxylic acid methyl ester,
3-{[(5-Chloro-thiophene-2-carbonyl)-amino]-methyl}-2,3-dihydro-indole-1,6-dicarboxylic acid 6-dimethylamide 1-{[2-fluoro-4-(2-oxo-2H-pyridin-1-yl)-phenyl]-amide},
5-Chloro-thiophene-2-carboxylic acid (1-{[2-fluoro-4-(2-oxo-2#H!-pyridin-1-yl)-phenylcarbamoyl]-methyl}-2,3-dihydro-1H-indol-3-yl)-amide,
3-{[(5-Chloro-thiophene-2-carbonyl)-amino]-methyl}-4-methyl-2,3-dihydro-indole-1-carboxylic acid [2-fluoro-4-(2-oxo-2H-pyridin-1-yl)-phenyl]-amide, or
4-Chloro-3-{[(5-chloro-thiophene-2-carbonyl)-amino]-methyl}-2,3-dihydro-indole-1-carboxylic acid (2-fluoro-4-(2-oxo-2H-pyridin-1-yl)-phenyl]-amide.

46. A pharmaceutical composition comprising a compound according to claim 1 and a pharmaceutically acceptable excipient.
